(19) United States
(12) Patent Application Publication
(10) Pub. No.: US 2026/0060567 A1
(43) Pub. Date: Mar. 5, 2026

(54) SYSTEMS AND METHODS FOR CAPTURING AEROSOLIZED BIOMATERIAL PARTICLES USING PACKED BEDS

(71) Applicant: Zeteo Tech, Inc., Sykesville, MD (US)

(72) Inventors: Dapeng CHEN, Sykesville, MD (US); Michael MCLOUGHLIN, Sykesville, MD (US); Wayne A. BRYDEN, Sykesville, MD (US)

(73) Assignee: Zeteo Tech, Inc., Sykesville, MD (US)

(21) Appl. No.: 19/379,528

(22) Filed: Nov. 4, 2025

Related U.S. Application Data
(63) Continuation-in-part of application No. PCT/US2024/030434, filed on May 21, 2024.
Application No. PCT/US2024/030434 is a continuation-in-part of application No. 18/532,476, filed on Dec. 7, 2023, now Pat. No. 12,364,411.
Application No. 18/532,476 is a continuation of application No. 17/916,649, now Pat. No. 11,839,463.
(60) Provisional application No. 63/005,179, filed on Apr. 3, 2020.
(60) Provisional application No. 63/010,029, filed on Apr. 14, 2020.
(60) Provisional application No. 63/069,029, filed on Aug. 22, 2020.
(60) Provisional application No. 63/496,307, filed on Apr. 14, 2023.
(60) Provisional application No. 63/715,996, filed on Nov. 4, 2024.

(51) Int. Cl. A61B 5/08; A61B 5/097; G01N 33/497

(52) U.S. Cl. CPC A61B 5/082; A61B 5/097; G01N 33/497

(57) ABSTRACT

Systems and methods for capturing and analyzing non-volatile organic aerosolized particles in exhaled breath. The systems and methods selectively capture aerosolized non-volatile organic particles in exhaled breath particles using an exhaled breath capture module including a packed bed column. The captured non-volatile organic particles are eluted using solvents and analyzed using analytical devices including MALDI-TOFMS.

### CROSS-REFERENCE TO RELATED APPLICATIONS

This patent application is related to and claims priority to U.S. Provisional Pat. Appl. No. 63/715,996 entitled “SYSTEMS AND METHODS FOR CAPTURING AEROSOLOZED BIOMATERIAL PARTICLES USING PACKED BEDS,” and filed on Nov. 4, 2024. This application is also a continuation-in-part application of International Pat. Appl. No PCT/US2024/030434 entitled “SYSTEMS AND METHODS FOR CAPTURING AEROSOLOZED BIOMATERIAL PARTICLES USING PACKED BEDS,” and filed on May 21, 2024, which is related to and claims priority to U.S. Provisional Pat. Appl. No. 63/469,307 entitled “SYSTEMS AND METHODS FOR CAPTURING AEROSOLOZED BIOMATERIAL PARTICLES USING PACKED BEDS,” and filed on May 26, 2023, and is a continuation-in-part application of U.S. patent application Ser. No. 18/532,476 entitled “DIAGNOSIS OF RESPIRATORY DISEASES USING ANALYSIS OF EXHALED BREATH AND AEROSOLS,” and filed on Dec. 7, 2023, now U.S. Pat. No. 12,364,411, which is a continuation of U.S. patent application Ser. No. 17/916,649 entitled “DIAGNOSIS OF RESPIRATORY DISEASES USING ANALYSIS OF EXHALED BREATH AND AEROSOLS,” and filed on Oct. 3, 2022, now U.S. Pat. No. 11,839,463, which is a U.S. National Stage Application of International Pat. Appl. No. PCT/US2020/048035 entitled “DIAGNOSIS OF RESPIRATORY DISEASES USING ANALYSIS OF EXHALED BREATH AND AEROSOLS,” filed on Aug. 26, 2020, which is related to and claims priority to U.S. Provisional Pat. Appl. No. 63/005,179 entitled “Diagnosis of Respiratory Diseases Using Exhaled Breath,” and filed on Apr. 3, 2020, and U.S. Provisional Pat. Appl. No. 63/010,029 entitled “Diagnosis of Respiratory Diseases Using Exhaled Breath,” and filed Apr. 14, 2020, and U.S. Provisional Pat. Appl. No. 63/069,029 entitled “Diagnosis of Respiratory Diseases Using Analysis of Exhaled Breath and Aerosols” and filed on Aug. 22, 2020. The disclosures of all prior applications are considered part of and are incorporated by reference in this patent application in their respective entireties.

### FEDERALLY SPONSORED RESEARCH AND DEVELOPMENT

None.

### TECHNICAL FIELD

This disclosure relates to methods and devices for capturing and analyzing aerosolized organic biomaterials such as virus and bacteria particles in exhaled breath. More particularly, but not by way of limitation, the present disclosure relates to methods and devices for capturing and analyzing non-volatile organics in exhaled breath and other aerosols to detect respiratory diseases such as COVID-19 and tuberculosis using mass spectromtery, including MALDI-TOFMS.

### BACKGROUND

Coronavirus Disease (COVID-19) is a disease caused by the newly emerged coronavirus SARS-COV-2. The novel coronavirus is a respiratory virus and spreads primarily through droplets generated when an infected person coughs or sneezes, or through droplets of saliva or discharge from the nose. The novel coronavirus is highly contagious and has created an ongoing COVID-19 pandemic which suggests that this virus is spreading more rapidly than influenza. To help in mitigation, rapid detection tools are needed.

Further, tuberculosis (TB) has surpassed HIV/AIDS as a global killer with more than 4000 daily deaths. (Patterson, B., et al., 2018). The rate of decline in incidence remains inadequate at a reported 1.5% per annum and it is unlikely that treatment alone will significantly reduce the burden of disease. In communities with highly prevalent HIV, Mycobacterium tuberculosis (Mtb) genotyping studies have found that recent transmission, rather than reactivation, accounts for the majority (54%) of incident TB cases. The physical process of TB transmission remains poorly understood and the application of new technologies to elucidate key events in infectious aerosol production, release, and inhalation, has been slow. Empirical studies to characterize airborne infectious particles have been sparse. Two major difficulties plaguing investigation are the purportedly low concentrations of naturally produced Mtb particles, and the complication of environmental and patient derived bacterial and fungal contamination of airborne samples. There have nonetheless been a number of attempts at airborne detection. A 2004 proof-of-concept study and subsequent feasibility study in Uganda sampled cough-generated aerosols from pulmonary TB patients. Coughing directly into a sampling chamber equipped with two viable cascade impactors resulted in positive cultures from more than a quarter of participants despite their having received 1-6 days of chemotherapy. A follow-up work employing the same apparatus found that participants with higher aerosol bacillary loads could be linked to greater household transmission rates and development of disease findings which suggest that quantitative airborne sampling may serve as a clinically relevant measure of infectivity. Therefore, interruption of transmission would likely have a rapid, measurable impact on TB incidence.

The best method to control transmission of tuberculosis is to promptly identify and treat active TB cases. (Wood, R. C., et al., 2015). Diagnosis of pulmonary TB is usually done by microbiological, microscopic, or molecular analysis of patient sputum. The “gold standard” test for TB infection in most of the developing world is a smear culture based on a sputum sample. The sample is smeared onto a culture plate, a stain is added that is specific to Mtb, and the stained cells are counted using a microscope. If the concentration of cells in the smear is greater than a set threshold, then the sample is classified as positive. If the TB counts are below this threshold, it is classified as negative. Diagnosis may take several hours. The need for sputum as a diagnostic sample is a limiting factor due to the challenges of collecting it from patients and to its complex composition. The viscosity of the material restricts test sensitivity, increases sample-to-sample heterogeneity, and increases costs and labor associated with testing. Moreover, sputum production (which requires coughing) is an occupational hazard for healthcare workers.

Sputum has several drawbacks as a sample medium. First, only about 50% of patients can provide a good sputum sample. For example, children under about age of eight often are not able to produce a sample upon request, usually because they have not developed an ability to “cough up” sputum from deep in their throat. The elderly and ill may not have the strength to cough up sputum. Others simply may not have sputum in their throat. Thus, a diagnostic method based on sputum analysis may not provide a diagnosis in as many as 50% of the patients who are in need of diagnosis. Sputum is also not useful as a diagnostic sample if it is collected one to two days after a person has been treated with antibiotics because the sample is no longer representative of the disease state deep in the lungs, and within several days after treatment begins, the number of live Mtb in the sputum is significantly reduced. Urine and blood have been proposed as sample media for the diagnosis of TB infection. Blood is highly invasive and entails the higher cost of handling blood samples that are often HIV positive since, in some parts of the world, many TB patients also have HIV co-infections. Further, a patient with an active TB infection may not have many TB cells circulating in their blood. Urine-based diagnostics have also been proposed, but these tests look for biomarkers of the disease other than living TB bacilli and have not been validated for widespread clinical use.

A sample that is easier, safer, and more uniform to collect and handle would simplify TB diagnosis. Exhaled breath contains aerosols (“EBA”) and vapors that can be collected noninvasively and analyzed for characteristics to elucidate physiologic and pathologic processes in the lung. (Hunt, 2002). To capture the breath for assay, exhaled air is passed through a condensing apparatus to produce an accumulation of fluid that is referred to as exhaled breath condensate (“EBC”). Although predominantly derived from water vapor, EBC has dissolved within its nonvolatile compounds, including cytokines, lipids, surfactant, ions, oxidation products, and adenosine, histamine, acetylcholine, and serotonin. In addition, EBC traps potentially volatile water-soluble compounds, including ammonia, hydrogen peroxide, ethanol, and other volatile organic compounds. EBC contains aerosolized airway lining fluid and volatile compounds that provide noninvasive indications of ongoing biochemical and inflammatory activities in the lung. Rapid increase in interest in EBC has resulted from the recognition that in lung disease, EBC has measurable characteristics that can be used to differentiate between infected and healthy individuals. These assays have provided evidence of airway and lung redox deviation, acid-base status, and degree and type of inflammation in acute and chronic asthma, chronic obstructive pulmonary disease, adult respiratory distress syndrome, occupational diseases, and cystic fibrosis. Characterized by uncertain and variable degrees of dilution, EBC may not provide precise assessment of individual solute concentrations within native airway lining fluid. However, it can provide useful information when concentrations differ substantially between health and disease or are based on ratios of solutes found in the sample.

Patterson et al. (2018) used a custom-built respiratory aerosol sampling chamber (RASC), a novel apparatus designed to optimize patient-derived exhaled breath aerosol sampling, and to isolate and accumulate respirable aerosol from a single patient. Environmental sampling detects the Mtb present after a period of ageing in the chamber air. 35 newly diagnosed GeneXpert (Cepheid, Inc., Sunnyvale, CA) sputum-positive, TB patients were monitored during one-hour confinement in the RASC chamber which has a volume of about 1.4 m³. The GeneXpert genetic assay is based on polymerase chain reaction (PCR) and may be used to analyze a sample for TB diagnosis and to indicate whether or not there are drug resistance genes in the TB sample. The GeneXpert PCR assay for TB can accept a sputum sample and provide a positive or negative result in about one hour. The chamber incorporated aerodynamic particle size detection, viable and non-viable sampling devices, real-time CO₂ monitoring, and cough sound-recording. Microbiological culture and droplet digital polymerase chain reaction (ddPCR) were used to detect Mtb in each of the bio-aerosol collection devices. Mtb was detected in 77% of aerosol samples and 42% of samples were positive by mycobacterial culture and 92% were positive by ddPCR. A correlation was found between cough rate and culturable bioaerosol. Mtb was detected on all viable cascade impactor stages with a peak at aerosol sizes 2.0-3.5 μm. This suggests a median of 0.09 CFU/liter of exhaled air for the aerosol culture positives and an estimated median concentration of 4.5×10⁷ CFU/ml of exhaled particulate bio-aerosol. Mtb was detected in bioaerosols exhaled by a majority of the untreated TB-patients using the RASC chamber. Molecular detection was found to be more sensitive than Mtb culture on solid media.

Mtb can be identified in EBA by culture, ddPCR, electron microscopy, immunoassay, and cell staining (e.g., oramine and dmn-Tre). Of these, PCR and immunoassays have the potential to be rapid and specific to the species level. PCR and other genomics-based techniques can be specific to the strain level. Mass spectrometry has also been shown to be specific to the strain level for cultures obtained from bacterial infections. For example, the Biotyper from Bruker Daltonics (Germany), has been shown to be able to identify up to 15,000 strains of bacteria that cause infections in humans. These techniques have been shown to be capable of identifying TB infection from EBA. Immunoassays for Mtb detection, such as the one based on lipoarabinomannan, are also well known.

In the case of TB, people infected with TB are often diagnosed through passive case finding when individuals present themselves to clinics. Active case finding (“ACF”) is generally considered to include other methods of reaching people suspected of TB infection outside of the primary health care system. According to WHO, ACF is “systematic identification of people with suspected active TB, using tests, examinations, or other procedures that can applied rapidly.” The goal of ACF is to get those infected to treatment earlier, reducing the average period of infection, and thereby reducing the spread of the disease. In the case of TB, by the time an individual goes to a clinic for help, that person may have transmitted the TB infection to between about 10 other people and about 115 other people. ACF can help to reduce or prevent significant TB transmission. The diagnostic systems and methods such as sputum analysis and blood analysis are either not automated and autonomously operated, or not rapid. Many have expensive assays that are consumed for each analysis, and thus, do not have general utility for active case finding, particularly in developing and under-developed countries. As previously described, EBA analysis appears to be a compelling diagnostic tool for TB detection that provides rapid analysis, portability, and low cost because the need for expensive assays and consumables are eliminated. McDevitt et al. (2013) have report EBA analytical devices and methods for influenza diagnosis. An impactor is used to remove large particles (>4 μm) from exhaled breath, followed by a wetted-film collector for the smaller particles (<4 μm). The two size bins of collected particles were analyzed for influenza virus using a genomics-based method, reverse transcriptase polymerase chain reaction (rt-PCR). PCR technology uses biomolecular probes, combined with other biomolecules including enzymes, to amplify a specific sequence of DNA if that particular sequence is present in the sample. The targeted sequences are believed to be specific to the disease being identified.

McDevitt et al. showed that EBA samples can be used to diagnose influenza. The disclosed devices and methods have several shortcomings from a practical standpoint. First, the breath aerosol sample is collected into discrete samples that are several milliliters in volume, and thus, considerable effort is needed to concentrate the sample. Further, the diagnostic device is not coupled to or integrated with the sample collector and is not amenable for use as an ACF tool. The ability to automate the RNA assays to create an autonomous diagnostic tool for TB analysis is not clear. A method to determine whether sufficient volume of cough or breath aerosol was generated by a particular patient is not described. As a result, if a sample is found to be negative for influenza it may be due to a false negative resulting from inadequate sample collection. It is well known that there are large variabilities among humans with respect to the volume of aerosolized lung fluid produced during various breathing maneuvers.

The GeneXpert Ultra is a state-of-the-art genomics-based point of care diagnostic device which uses PCR technology. It may be integrated with an EBA sample collection method to perform ACF of TB and other respiratory diseases, but the sample collection times would be too long to be practical. Patterson et al. have shown that between 20 and 200 TB bacilli are typically produced in EBA and can be collected over a one-hour sampling period. A minimum of one hour of sampling would be required to use the GeneXpert Ultra as a diagnostic assay. The GeneXpert device may be integrated with a system that samples air to analyze air samples for airborne pathogens. The BDS system (Northup Grumman, Edgewood, MD), is being used for screening US Postal Service mail for bacterial spores that cause anthrax as the mail passes through distribution centers. It combines a wetted-wall cyclone with a GeneXpert PCR system to autonomously sample air and report if pathogens are present. However, the GeneXpert Ultra assay has a relatively high cost per test and takes approximately an hour to complete the assay and provide a result. In general, PCR-based diagnostics are unsuitable for TB screening for ACF applications due to both the extended time needed for sampling and analysis, and the relatively high cost per test.

The time associated with a diagnostic assay is a critical parameter for a fielded, or “point of care” test. ACF is an example of a fielded diagnostic assay because, by definition, ACF takes place outside the healthcare system. In the U.S., a point-of-care test needs to provide an answer in 20 minutes or less. If not, the test is considered to be too slow and not acceptable for achieving short patient wait-times. In the developing world, and especially in countries with a history of TB prevalence, the GeneXpert device may be used to provide diagnosis in about one hour. As previously described, this assay is expensive to implement on a “cost per test” basis, and therefore it is not yet widely deployed. Because of its high cost, it is not used to screen patients who appear healthy (non-symptomatic) but might have TB infection, but rather, is used to confirm a diagnosis that is strongly suspected based on other tests or factors.

Fennelly et al. (2004) described TB analysis using cough aerosol and a collection chamber that contains two Anderson cascade impactors using individuals who were known to have active patients. Individuals were asked to provide two discrete five-minute bursts of intense coughing. Culturing of impacted samples took 30-60 days, and therefore this approach is not amenable to automation. A challenging aspect of EBA as a clinical sample is the relatively small sample of volume of exhaled particulates that can be collected from breath. Further, a significant fraction of the mass collected is water. The molecules that contain diagnostic information (“biomarkers”) are present in nanoliter or picogram quantities. Subsequently, the aerosol collection method must be effective in capturing a large fraction of the biomass in the exhaled breath. Exhaled breath includes air that is exhaled from the lungs through any number of maneuvers, including tidal breathing, deep breathing, coughing, and sneezing. Particular types of deep breathing maneuvers such as forced vital capacity (FVC), may be used to measure the maximum volume of lung capacity by breathing in as much as possible, and exhaling as far (or as deep) as possible to maximize the volume of exhaled breath. Forced expiratory volume (FEV) measures how much air a person can exhale during a forced breath. The amount of air exhaled may be measured during the first (FEV1), second (FEV2), and/or third seconds (FEV3). Forced vital capacity (FVC) is the total amount of air exhaled during an FEV test. Forced expiratory volume and forced vital capacity are lung function tests that are measured during spirometry. Forced expiratory volume is an important measurement of lung function.

Although research has shown that respiratory diseases can be detected from breath aerosol and breath condensate, modern clinical tests for infections or diseases such as tuberculosis, influenza, pneumonia continue to utilize sputum, blood, or nasal swabs. Exhaled breath analytical tools have not been commercialized because methods and devices to efficiently collect and concentrate the trace amounts of analyte present in exhaled breath are lacking. Furthermore, there is no standard or methodology to assess how much exhaled breath is sufficient for a particular diagnosis. The disclosed example devices and methods overcome these limitations by collecting exhaled breath aerosol and breath condensate at high flow rate, high efficiency, and into relatively concentrated samples. Further, size sorting of aerosol can be incorporated to increase the signal to noise ratio for specific analytes prior to collection of the analytes. The concentrated samples may then be analyzed by several methods, but preferably, using methods that are sensitive, rapid, and highly specific to the analytes of interest. Preferably, the analysis will be rapid, and near real-time. Mass spectrometry, real-time PCR, and immunoassays have the highest potential to be sensitive, specific and nearly real-time.

A need exists for sample collection methods that can be coupled with fast diagnostic tools such as mass spectrometry (“MS”) that is more rapid and reliable than sputum analysis and less invasive than blood analysis to provide a diagnostic assay that is fast, sensitive, specific and preferably, characterized by low cost per test. Such a system could be used for active case finding (ACF) of TB and other lung or respiratory tract diseases. To be effective, a system for ACF must be rapid and inexpensive on a “per diagnosis” basis. Low cost-per-test is a requirement for screening a large number of individuals to proactively prevent TB transmission to search for the few that are indeed infected with TB. Low-cost devices and methods would also be required for point-of-care diagnosis of influenza and other pathogenic viruses because patients probably infected with a “common cold” may be infected with rhinovirus. In some cases, the respiratory infection will be driven by a bacterial or fungal microbe and may be treatable with antibiotics. In other cases, the microbe may be resistant to antibiotics, and a diagnostic method that can identify microbial resistance to antibiotics is preferable. Rapid EBA methods for distinguishing between viral and bacterial infections in the respiratory tract are desired while minimizing the occurrence of false negatives due to an insufficient sample volume. Mass spectrometry, genomics methods including PCR, and immunoassays have the highest potential to be sensitive and specific. Mass spectrometry, and in particular, MALDI time-of-flight mass spectrometry (MALDI-TOFMS), is a preferred diagnostic tool for analysis EBA and EBC samples because it has been demonstrated to be sensitive, specific and near real-time.

### BRIEF DISCLOSURE

This summary is provided to introduce in a simplified form a selection of concepts that are further described below in the Detailed Description. This Summary is not intended to identify key features or essential features of the claimed subject matter, nor is it intended to limit the scope of the claimed subject matter.

In some implementations, a passive exhaled breath aerosol particle capture system for capturing non-volatile organic particles in exhaled breath may include an exhaled breath capture module, a passive CO₂ sensor disposed downstream of the exhaled breath capture element, and one or more air filters removably connected to one or more of the pressure relief port disposed in the module or disposed downstream of the CO₂ sensor. In some instances, the exhaled breath capture module to selectively capture non-volatile organic particles in exhaled breath may include an exhaled breath management chamber including a chamber inlet end configured to receive an exhaled breath tubing removably insertable into the chamber, a chamber outlet end, and a pressure relief port disposed near the chamber outlet end, and an exhaled breath capture element including a packed bed column, the capture element removably connected to the chamber outlet end. In some examples, a gap of predetermined length may be defined between an outlet end of the exhaled breath tubing when the exhaled air tubing is positioned in the exhaled breath management chamber and the inlet end of the exhaled breath capture element. The non-volatile organic particles may include one or more of metabolite biomarkers, lipid biomarkers, proteomic biomarkers, bacteria particles, or virus particles characteristic of one or more respiratory diseases.

In some implementations, the passive CO₂ sensor may include a colorimetric sensor. In some instances, exhaled breath may be drawn into the packed bed column without using a pump disposed downstream of the exhaled breath capture element. Instead, exhaled breath may be forced into the packed bed column when an individual breathes into the exhaled breath tubing. Accordingly, an example exhaled breath capture module may be a passive module and be configured to capture non-volatile organic particles in exhaled breath without requiring electrical power.

In some other implementations, the exhaled breath flow rate through the exhaled breath capture element may be between about 0.5 L/min and about 10 L/min. In some instances, the packed bed column in exhaled breath capture element may be maintained in a moist or wet state prior to exhaled breath capture. In some other instances, an example packed bed column may include beads having an average diameter of between about 40 μm and about 120 μm. In some instances, an example exhaled breath aerosol particle capture module may be defined by an exhaled breath aerosol particle capture efficiency of greater than 99%. In some other instances, a pressure drop across the exhaled breath capture element may be less than about 100 mbar.

In some instances, the outlet end of the capture element may be disposed outside the exhaled breath management chamber. In some other instances, the chamber may include an adapter disposed at the inlet end of the chamber, the adapter defining an annular region between the adapter and the chamber. In some examples, the exhaled air tubing may be removably inserted into the chamber through the chamber inlet end and through the adapter.

In some implementations, the adapter may include a recess element configured to receive the outlet end of the exhaled air tubing. The recess element may be configured to define the gap between the outlet end of the exhaled breath tubing and the inlet end of the exhaled breath capture element. In some instances, the exhaled breath tubing may be made of one or more of paper, plastic, or metal. In some implementations, a nominal diameter of the inlet end of the exhaled breath capture element inlet may be substantially equal to a diameter of the exhaled breath tubing. In some instances, the chamber inlet end may include a removable chamber cap. The example adapter may be removable from the chamber by opening the cap.

In some implementations, the pressure relief port may be disposed orthogonal to a longitudinal axis (A-A′) of the exhaled breath capture element when the capture element is removably connected to the chamber outlet end. In some other implementations, an example exhaled breath capture module may further include an instrument port disposed at the chamber outlet end.

In some implementations, an example packed bed column may include solid particles or beads including one or more of resins, cellulose, silica, agarose, or hydrated Fe₃O₄ particles. In some instances, the packed bed column may include resin beads having C18 functional groups on the surface. In some other instances, an example packed bed column may include cellulose beads having sulfate ester functional groups on the surface. In some instances, the beads in an example packed bed column may have an average diameter of between about 20 μm and about 500 μm. In some other instances, the beads may be packed between two porous polymeric frit discs.

In some implementations, the beads in an example packed bed may be functionalized with one or more functional groups including immobilized on the surface of the particles, wherein the functional groups include at least one of C18 (octadecyl), octyl, ethyl, cyclohexyl, phenyl, cyanopropyl, aminopropyl, 2,3-dihydroxypropoxypropyl, trimethyl-aminopropyl, carboxypropyl, benzenesulfonic acid, propylsulfonic acid, an ion exchange phase, a polymer phase, antibodies, glycans, lipids, DNA, or RNA. In some instances, the ion exchange phase may include one or more of diethylaminoethyl cellulose, QAE Sephadex, Q sepharose, or carboxymethyl cellulose. In some other instances, the polymer phase may include one or more of polystyrene-co-1,4-divinylbenzene, methacrylates, polyvinyl alcohol, starch, or agarose. In some aspects, antibodies may include one or more of anti-human albumin, anti-Influenza A virus NP, or Anti-SARS-COV-2 virus NP.

In some other implementations, an example exhaled breath aerosol particle capture system may include any one of the exhaled breath capture modules previously described herein. Additionally, an example exhaled breath aerosol particle capture system may include a pump removably connected to the outlet end of the exhaled breath capture element, a CO₂ sensor disposed upstream of the pump, a particle counter removably connected to an instrument port disposed at the chamber outlet end, an air filter removably connected to the pressure relief port, a microcontroller configured to control the operation of the exhaled breath aerosol particle capture system, and a power supply configured to supply power to the exhaled breath aerosol particle capture system.

In some implementations, an example exhaled breath aerosol particle capture system may further include a saliva or breath condensate cup disposed upstream of the air filter. In some instances, an example exhaled breath aerosol particle capture system may be defined by an exhaled breath aerosol particle capture efficiency of greater than 99%. In some other instances, the pump may be configured to draw exhaled breath through the exhaled breath capture element at a flow rate of between about 0.5 L/min and about 10 L/min.

In some implementations, an example system for analyzing non-volatile organic particles in exhaled breath may include any one of the exhaled breath aerosol particle capture systems previously described herein, an extraction system configured to extract the captured non-volatile organic particles from the packed bed column in the exhaled breath capture element into one or more liquid samples, and an analytical device to analyze the non-volatile organic particles in the one or more liquid samples. In some instances, the extraction system may include means to flush the packed bed column with at least one solvent and produce the one or more liquid samples. In some other instances, example analytical devices may include one or more of PCR, ELISA, rt-PCR, mass spectrometer (MS), MALDI-MS, ESI-MS, MALDI-TOFMS, or LC-MS/MS.

In some implementations, a method for analyzing non-volatile organic particles in exhaled breath may include capturing non-volatile organic particles in exhaled breath using any one of the exhaled breath aerosol particle capture systems previously described herein, extracting the non-volatile organic particles from the packed bed column into one or more liquid samples by flushing the packed bed column with one or more solvents, and analyzing the aerosol particles in the one or more liquid samples to determine the presence or absence of the respiratory disease. In some examples, the example method may further include maintaining the packed bed column in a wet state prior to capturing the non-volatile organic particles in the exhaled breath.

In some instances, the one or more solvents may include one or more of acetonitrile (“ACN”), methanol, trifluoro acetic acid (“TFA), or isopropanol (IPA”), the remaining being water. In some other instances, the one or more solvents may include one or more of between about 50 vol % and about 70 vol % acetonitrile in water, between about 50 vol % and about 70 vol % isopropanol in water, or between about 0.05 vol.-% TFA in water. In some instances, the extracting operation may include flushing the packed bed column using one or more of about 70 vol % ACN in water, water, or about 0.05 vol % TFA in water. In some other instances, the extracting operation may include flushing the packed bed column first with about 50 vol % ACN in water and subsequently with about 70 vol % IPA in water.

In some implementations, an example method for analyzing non-volatile organic particles in exhaled breath may further include sample processing including mixing the one or more liquid samples with a MALDI matrix and applying the one or more mixed samples to one or more sample plates. In some other implementations, an example method for analyzing non-volatile organic particles in exhaled breath may further include sample processing including subjecting the one or more liquid samples to protein digestion and generating a peptide sample characteristic of the respiratory disease. In some instances, the analyzing the sample, including one or more of analyzing the aerosol particles in the one or more liquid samples, or the samples after sample processing, may include using one or more of PCR, ELISA, rt-PCR, mass spectrometer (MS), MALDI-MS, ESI-MS, MALDI-TOFMS, or LC-MS/MS.

In some implementations, an exhaled breath capture element for selectively capturing non-volatile organic particles in exhaled breath may include a packed bed column disposed between an exhaled breath capture element inlet end and an exhaled breath capture element outlet end. In some instances, an example packed bed column may include a first packed bed including silica gel beads, wherein the first packed bed is disposed proximate to the exhaled breath capture element inlet end, and a second packed bed including resin beads functionalized with C18 groups, wherein the second packed bed is disposed downstream of the first packed bed. In some instances, an example packed bed column may be saturated with water and sealed by closing the exhaled breath capture element inlet end and the exhaled breath outlet end. In some other instances, the silica gel beads may have an average diameter of between about 20 μm and about 500 μm. In some other instances, the resins beads may be functionalized with C18 groups and have an average diameter of between about 20 μm and about 500 μm. In some instances, the first packed bed and second packed bed may each be packed between porous polymeric frit discs. In some examples, the packed bed column may be maintained in a wet state prior to capturing the non-volatile organic particles in the exhaled breath.

In some implementations, an exhaled breath capture element for selectively capturing non-volatile organic particles in exhaled breath may include a packed bed column disposed between an exhaled breath capture element inlet end and an exhaled breath capture element outlet end. In some instances, an example packed bed column may include a first packed bed including beads functionalized with one or more of aminopropyl (NH₂), diols, cyanopropyl, sulfonic acid, carboxylic acid, or polyethyleneimine groups, wherein the first packed bed is disposed proximate to the exhaled breath capture element inlet end, and a second packed bed including beads functionalized with one or more of C18, C8, C4, C30, or phenyl groups, wherein the second packed bed is disposed downstream of the first packed bed. In some instances, an average diameter of the beads in the first packed bed may be greater than the average diameter of beads in the second packed bed. In some other instances, the beads in one or more of the first packed bed or the second packed bed may include one or more of silica, resins, cellulose, silica, agarose, or hydrated Fe₃O₄ particles. In some examples, the packed bed column may be maintained in a wet state prior to capturing the non-volatile organic particles in the exhaled breath.

Details of one or more implementations of the subject matter described in this disclosure are set forth in the accompanying drawings and the description below. Other features, aspects, and advantages will become apparent from the description, the drawings, and the claims. Note that the relative dimensions of the following figures may not be drawn to scale.

### BRIEF DESCRIPTION OF THE DRAWINGS

FIG. 1 shows a schematic diagram of an example exhaled breath sample collection system including a packed bed column, according to some implementations.

FIG. 2 shows a schematic diagram of an example diagnostic system for respiratory diseases including a sample collection system, according to some implementations.

FIG. 3 shows a schematic diagram of an example diagnostic method using a system that includes a packed bed column, according to some implementations.

FIG. 4 shows a schematic diagram of an example exhaled breath sample collection system including a packed bed column, according to some implementations.

FIGS. 5A-B show schematic diagrams of an example exhaled breath capture module including a packed bed column, according to some implementations.

FIG. 6A shows a schematic diagram of an example exhaled breath aerosol particle capture system, according to some implementations.

FIG. 6B shows a schematic diagram of an example exhaled breath aerosol particle capture system, according to some implementations.

FIG. 6C shows a schematic diagram of an example exhaled breath aerosol particle capture system, according to some implementations.

FIG. 7 shows a schematic diagram of an example aerosol sample collection test system including a packed bed column, according to some implementations.

FIG. 8A shows direct infusion mass spectrometry results depicting ion intensities of MS2 capsid protein in the control sample, according to some implementations.

FIG. 8B shows direct infusion mass spectrometry results depicting ion intensities of MS2 capsid protein in the captured aerosol sample, according to some implementations.

FIG. 9A shows nanoflow-LC mass spectrometry selected ion chromatogram of MS2 capsid protein in a control sample, according to some implementations.

FIG. 9B shows nanoflow-LC mass spectrometry selected ion chromatogram of MS2 capsid protein in a captured aerosol sample, according to some implementations.

FIG. 10A shows test results depicting the effect of C18 bead size on flow rate through an example sample capture element, according to some implementations.

FIG. 10B shows test results depicting the effect of bead quantity on flow rate through an example sample capture element, according to some implementations.

FIG. 10C shows test results depicting the effect of inlet polymeric frit pore size on flow rate through an example sample capture element, according to some implementations.

FIG. 11 shows protein blue staining results of aerosolized BSA proteins captured using an example C18 packed bed column sample capture element and a button sampler, according to some implementations.

FIG. 12A shows MALDI TOF mass spectra of hot acid digested peptides in culture media of SARS-COV-2 virus sample not treated with example sample capture element, according to some implementations.

FIG. 12B shows MALDI TOF mass spectra with peak assignments of hot acid digested peptides of SARS-COV-2 virus particles extracted from an example sample capture element, according to some implementations.

FIG. 12C shows distribution of peptide ion intensities constructed using bottom-up proteomics of SARS-COV-2 virus particles, according to some implementations.

FIG. 12D shows distribution of peptide ion intensities constructed using bottom-up proteomics of SARS-COV-2 virus particles extracted from an example sample capture element, according to some implementations.

FIG. 12E shows peptide mass fingerprint of SARS-COV-2 virus particles extracted from an example sample capture element using Mascot database search using different allowed missed site cleavage numbers, according to some implementations.

FIG. 13A shows a schematic diagram of another example exhaled breath aerosol particle capture system, according to some implementations.

FIG. 13B shows a schematic diagram of a passive exhaled breath aerosol particle capture system, according to some implementations.

FIG. 13C shows a schematic diagram of another passive exhaled breath aerosol particle capture system, according to some implementations.

Like reference numbers and designations in the various drawings indicate like elements.

### DETAILED DESCRIPTION

The following description is directed to some example implementations for the purpose of describing the innovative aspects of this disclosure. However, a person having ordinary skill in the art will readily recognize that the teachings herein can be applied in a multitude of different ways. The described implementations can be implemented in batteries for a variety of applications and may be tailored to compensate for various performance related deficiencies. As such, the disclosed implementations are not to be limited by the examples provided herein, but rather encompass all implementations contemplated by the attached claims. Additionally, well-known elements of the disclosure will not be described in detail or will be omitted so as not to obscure the relevant details of the disclosure.

Various aspects of the novel compositions and methods are described more fully herein with reference to the accompanying drawings. These aspects are provided so that this disclosure will be thorough and complete, and will fully convey the scope of the disclosure to those skilled in the art. Although some examples and aspects are described herein, many variations and permutations of these examples fall within the scope of the disclosure. Although some benefits and advantages of the various aspects are mentioned, the scope of the disclosure is not intended to be limited to benefits, uses, or objectives. The detailed description and drawings are merely illustrative of the disclosure rather than limiting, the scope of the disclosure being defined by the appended claims and equivalents thereof.

In this disclosure, aerosol generally means a suspension of particles dispersed in air or gas. “Autonomous” diagnostic systems and methods mean generating a diagnostic test result “with no or minimal intervention by a medical professional.” The U.S. FDA classifies medical devices based on the risks associated with the device and by evaluating the amount of regulation that provides a reasonable assurance of the device's safety and effectiveness. Devices are classified into one of three regulatory classes: class I, class II, or class III. Class I includes devices with the lowest risk and Class III includes those with the greatest risk. All classes of devices as subject to General Controls. General Controls are the baseline requirements of the Food, Drug and Cosmetic (FD&C) Act that apply to all medical devices. In vitro diagnostic products are those reagents, instruments, and systems intended for use in diagnosis of disease or other conditions, including a determination of the state of health, in order to cure, mitigate, treat, or prevent disease or its sequelae. Such products are intended for use in the collection, preparation, and examination of specimens taken from the human body. The example devices disclosed herein can operate and produce a high-confidence result autonomously, and consequently, have the potential to be regulated as a Class I device. In some regions of the world with high burdens of TB infection, access to medically trained personnel is very limited. An autonomous diagnostic system is preferred to one that is not autonomous.

Breath aerosol particles contain a variety of nonvolatile organic biomolecules such as metabolites, lipids, and proteins. Further, the nonvolatile molecules have a wide particle size distribution ranging from a sub-micron size to about 10 microns in size. Breath collection and disease diagnostic systems and methods that can efficiently capture different types of nonvolatile molecules of different particle sizes from exhaled breath are required. Particular aspects of the invention are described below in considerable detail for the purpose of illustrating the compositions, principles, and operations of the disclosed methods and systems. However, various modifications may be made, and the scope of the invention is not limited to the example aspects described.

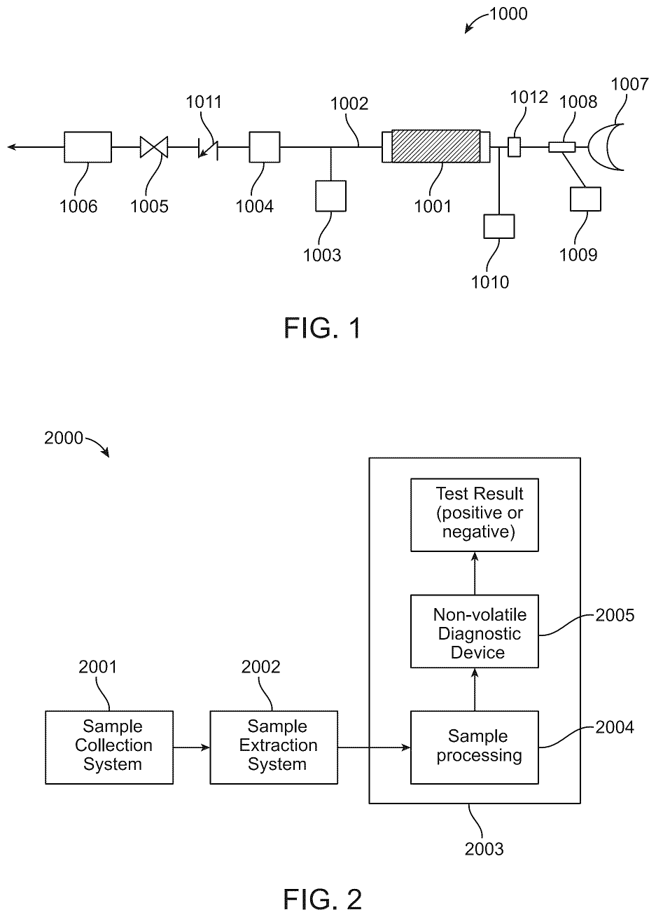

FIG. 1 shows a schematic diagram of an example exhaled breath sample collection system including a packed bed column, according to some implementations. Example exhaled breath sample collection system 1000 may include a sample capture element 1001 including a packed bed column to selectively capture breath aerosol including nonvolatile organisms including, but not limited to bacteria and viruses, and molecules including small molecules, lipids, and proteins on very high efficiency adsorbent materials. A trap 1003 may be disposed in fluid communication with column 1001 using tubing 1002. Trap 1003 may be made of glass or plastic material. Trap 1003 may be cooled to below ambient temperature using an ice bath or other suitable means. Trap 1003 may be used to collect water vapor, other volatile (check) and nonvolatile molecules that may pass through the collection column as exhaled breath condensate (EBC). During breath analysis of a normally breathing person, element 1001 may be removably connected to a mouthpiece (not shown) into which the patient is instructed to breathe or otherwise execute a breath maneuver previously disclosed herein. For example, capture element 1001 may be removably connected downstream (at the outlet) of a breath collection element 1007 such as a first aid CPR rescue mask (e.g., as supplied by Dixie USA EMS Supply Co., Model Number EVR-CPR01) worn by the patient during breath analysis. A flow splitter 1008 may be disposed between breath collection element 1007 and capture element 1001 to divide the flow of exhaled breath such that a first portion of exhaled breath is directed to capture element 1001 and a second portion towards a HEPA filter 1009. Flow splitter 1008 may be integrated into collection element 1007.

In some implementations, a large-particle trap 1012 may be disposed upstream of capture element 1001 to remove large particles of breath condensate (greater than about 10 μm) from the exhaled breath stream prior to entering capture element 1001. Pump 1006 may be used to pull exhaled breath into the packed bed column in capture element 1001. An example pump 1006 is a portable diaphragm pump (e.g., Parker Hannifin Corp., Part No.: D737-23-01). The flow rate out of pump 1006 may be adjusted using needle valve 1005 to achieve a desired flow rate. Check valve (one-way flow valve) 1011 may be disposed between pump 1006 and capture element 1001 and is configured to be in an open position only when pump 1006 is pulling exhaled breath through the packed bed column. When there is no flow, valve 1011 is disposed in a closed position. A nominal flow rate of between about 200 ml/min and 600 ml/min may be used. In addition, several capture elements 1001 may be used in parallel to increase the flow rate up to 12 L/min.

In some implementations, when one or more capture elements are in collection mode, one or more may be in eluting mode, and the some may be in standby mode. To determine if exhaled breath sample volume was adequate, a CO₂ sensor and particle counter (not shown) may be disposed between breath collection element 1007 and sample capture element 1001. CO₂ monitoring and particle count allows for an approximation of the proportion of exhaled air volume. A HEPA filter may also be disposed downstream of trap 1003. Capture element 1001 may be cooled using a cooling jacket or other means to reduce the temperature to below ambient temperature to increase the collection efficiency of non-volatile organics particles. The breath sample collection system may further include a humidifier 1010 disposed upstream of the inlet to the capture element to humidify exhaled breath and increase the humidity in the packed bed column.

In some implementations, breath collection element 1007 may include a tight-fitting mask configured to receive an individual's face and may be removably attached using straps and the like to the face/head of a patient/individual. The individual may sit in an optional containment booth to isolate the patient's EBA from the ambient air in the testing room or area. Element 1007 may be used to collect and direct breath aerosol particles emitted though the mouth and nose of patient into capture element 1001 using pump 1006 as previously described without depositing the aerosol particles on the walls of element 1007. Element 1007 may be disposable to limit the risk of a patient becoming contaminated or infected with a pathogen emitted by a previous patient. Alternatively, element 1007 may be reusable, in which case it may be sterilized.

In some implementations, the example packed bed column in capture element 1001 may include Hamilton PRP-C18 resin beads as supplied by Sigma Aldrich and other vendors. The bed may be held in place between two porous filter plates such as frit discs. For example, a polyethylene disc having an average pore size of above 35 μm may be placed upstream of the bed and a polyethylene disc having an average pore size of 10 μm (Boca Scientific, Dedham, MA) may be placed downstream of the bed. The 35 μm frit disc allows a faster air flow rate while the smaller 10 μm frit disc traps all the C18 resin well. In an example capture element 1001, the packed bed may include about 25 mg of C18 resin beads having a nominal diameter between about 12 μm and about 20 μm. Non-volatile organic components in exhaled breath removably interact with the C18 functional groups on the beads and are trapped. Water, volatiles and other hydrophilic molecules pass through the bed and may be trapped in glass trap 1003.

In some implementations, in addition to C18 functional groups, other functional groups that show affinity to nonvolatile molecules may be used as adsorbents in the column immobilized on solid phase beads such as resin beads. The solid phase beads may be made of polymers and particles such as resins, cellulose, silica, agarose, and hydrated Fe₃O₄ nanoparticles. Adsorbent materials may include other functional groups that include, but are not limited to, octadecyl, octyl, ethyl, cyclohexyl, phenyl, cyanopropyl, aminopropyl, 2,3-dihydroxypropoxypropyl, trimethyl-aminopropyl, carboxypropyl, benzenesulfonic acid, and propylsulfonic acid disposed on solid phase beads. Functional groups may also include at least one of ion exchange phases, polymer phases, antibodies, glycans, lipids, DNA and RNA.

FIG. 2 shows a schematic diagram of an example diagnostic system 2000 for respiratory diseases including a sample collection system, according to some implementations. Example diagnosis system 2000 may include a breath sample collection system 2001 disposed in fluid communication with a sample extraction system 2002 and an analysis system 2003. Sample collection system 2001 may include the example sample collection system 1000, as previously described. Sample extraction system 2002 may be used to extract the trapped non-volatile organics from the packed bed column in system 1000 and may be disposed in-line or off-line in system 2000. When system 2002 is disposed off-line, at the conclusion of exhaled breath sample collection, capture element 1001 may be removed from system 1000 and eluted with an organic solvent in extraction system 2002 to remove non-volatile organics from the packed bed column.

In some implementations, example organic solvents may include, but are not limited to, about 50-70% acetonitrile in water to extract trapped non-volatile organics (strongly polar non-volatile organic molecules, proteins and the like) from the packed bed column. Extraction may be repeated using the same or another solvent, that includes, but is not limited to, 50-70% isopropanol in water to extract less polar lipid molecules from the packed bed. Other organic solvents include between about 50% and about 70% methanol in water, and about 50% methanol in about 50% chloroform.

In some implementations, when system 2002 is disposed in-line in diagnostic system 2000, at least one of a CO₂ sensor and particle counter may be disposed upstream of extraction system 2002. System 2002 may include a solvent vessel, a pump to transfer the solvent from the solvent to packed bed column and a vessel to collect the solvent including the non-volatile biomarkers into another vessel or cup. Alternately, system 2002 may include an injector to inject solvent into the packed bed column and collect the extract liquid including non-volatile organics and biomarkers in a suitable cup or vessel, or other laboratory tubes having a small volume. The captured sample in solvent may be further processed and analyzed in analysis system 2003.

In some implementations, analysis system 2003 may include sample processing system 2004 and at least one diagnostic device 2005. Sample processing system 2004 may include elements or components necessary to perform one or more of the following steps:
(a) Placing the sample in at least one of a cup, a vial and a sample plate. For example, the Series 110A Spot Sampler (Aerosol Devices) uses 32 well plates with circular well shape (75 μL well volume) or teardrop well shape (120 μL well volume) which are heated to evaporate the solvent and excess fluid/liquid in the sample to concentrate the sample;
(b) Placing the sample in a cup and exposed to a source of vacuum or freeze-drying device to cause the solvent to evaporate to concentrate the sample; and,
(c) hot digestion of proteins and virus particles.

In some implementations, the samples may be centrifuged to remove chemical contamination particles. Many diagnostic devices 2005 may be adapted for use in analysis system 2003 that include, but are not limited to, devices that perform genomics-based assays (such as PCR, rt-PCR and whole genome sequencing), biomarker recognition assays (such as ELISA), and spectral analysis such as mass spectrometry (“MS”). Of these diagnostic devices, MS is preferable on account of its speed of analysis. The MS techniques that are preferable for biomarker identification are electrospray ionization (ESI) and matrix assisted laser desorption ionization (“MALDI”) time of flight MS (TOFMS). ESI may be coupled to high resolution mass spectrometers.

MALDI-TOFMS devices may be compact, lightweight, consume less than 100 watts of power and provide sample analysis in less than 15 minutes. MALDI-TOFMS is a preferred diagnostic device for point-of-care diagnostics suitable for ACF. The sample must be dry before it is inserted into the vacuum chamber of the MS and subjected to laser pulses from an ultraviolet laser. This interaction between the sample and the laser creates large, informative biological ion clusters that are characteristic of the biological material. When a concentrated sample is provided by sample processing system 2004 including only trace levels of water or trace levels organic solvents such as 50% to 70% of one of acetonitrile, methanol, or isopropanol in water, sample analysis using MS may take less than 5 minutes (including the sample preparation) because less time is needed to evaporate the water from the sample.

In some implementations, MALDI-TOFMS may be used to identify live/active agents that include one or more of B. anthracis spores (multiple strains), Y. pestis, F. tularensis, Venezuelan equine encephalitis virus (VEE), Western equine encephalomyelitis virus (WEE), Eastern equine encephalitis virus (EEE), botulinum neurotoxins (BoNT), staphylococcus Enterotoxin (SEA), Staphylococcal enterotoxin B (SEB), ricin, abrin, Ebola Zaire strain, aflatoxins, saxitoxin, conotoxins, Enterobacteria phage T2 (T2), HT-2 toxins (HT2), cobra toxin, biothreat simulants including B. globigii spores, B. cereus spores, B. thuringiensis Al Hakam spores, B. anthracis Sterne spores, Y. enterocolitica, E. coli, MS2 virus, T2 virus, Adenovirus and nonvolatile biochemical threats including NGAs (nonvolatile), bradykinin, oxytocin, Substance P, angiotensin, diazepam, cocaine, heroin, or fentanyl. Further, the example systems and methods disclosed herein may be used to achieve accurate detection and identification of SARS-CoV-2 from human breath samples.

In MALDI-MS analysis, the target particle (analyte) is coated by a matrix chemical, which preferentially absorbs light (often ultraviolet wavelengths) from a laser. In the absence of the matrix, the biological molecules would decompose by pyrolysis when exposed to a laser beam in a mass spectrometer. The matrix chemical also transfers charge to the vaporized molecules, creating ions that are then accelerated down a flight tube by the electric field. Microbiology and proteomics have become major application areas for mass spectrometry; examples include the identification of bacteria, discovering chemical structures, and deriving protein functions. MALDI-MS has also been used for lipid profiling of algae. During MALDI-MS, a liquid, usually included an acid, such as trifluoroacetic acid (TFA), and a MALDI matrix chemical such as alpha-cyano-4-hydroxycinnamic acid, is dissolved in a solvent and added to the sample. Solvents include one or more of acetonitrile, water, ethanol, or acetone. TFA is normally added to suppress the influence of salt impurities on the mass spectrum of the sample. Water enables hydrophilic proteins to dissolve, and acetonitrile enables the hydrophobic proteins to dissolve.

In some implementations, the MALDI matrix solution is spotted on to the sample on a MALDI plate to yield a uniform homogenous layer of MALDI matrix material on the sample. The solvents vaporize, leaving only the recrystallized matrix with the sample spread through the matrix crystals. The acid partially degrades the cell membrane of the sample making the proteins available for ionization and analysis in an MS. Other MALDI matrix materials include 3,5-dimethoxy-4-hydroxycinnamic acid (sinapinic acid), α-cyano-4-hydroxycinnamic acid (α-cyano or α-matrix) and 2,5-dihydroxybenzoic acid (DHB), as described in U.S. Pat. No. 8,409,870.

Additionally, the volatile organic compounds collected in trap 1003 (FIG. 1) may be warmed using a heater, to drive off the volatile compounds into a diagnostic device such as GC-MS, GC-IMS, volatile ion chromatography, or any other type of analysis method suitable for analyzing volatile organic compounds.

Virus (e.g., SARS-COV-2) detection is centered on detection of viral proteins, which is a difficult challenge. An example method for virus detection may include a glycan-based capture matrix (beads) to pull the target virus out of the background matrix (e.g., other non-virus biomolecule contaminants). An aliquot of the sample which may contain a virus, for example, collected using sample collection system 1000, which may also include other background contaminants, may be applied to a bead carrying the capture probe. At least one of glycan, heparin, and carbohydrates may be used as capture materials or probes bound on resin beads or some other types of beads. An optional washing step may be used to remove any nontargeted-virus contaminants.

In some implementations, the concentrated and purified virus may be eluted off the beads in the packed bed column in capture element 1001 using suitable solvents into a sealed heating chamber containing an organic acid which may include formic acid or acetic acid and heated to 120° C. for about 10 minutes to digest the proteinaceous toxin down into specific peptide fragments. This hot acid protein digestion protocol cleaves the protein at aspartic acid residues creating a highly reproducible peptide pattern. The capture and digestion processes described may be accomplished with antibodies and enzymes, respectively. Using this example sample processing method for MALDI-TOFMS, sensitivity for ricin biotoxin of better than 100 ng/ml (with S/N of about 50:1) in clean buffer may be achieved. At S/N (signal to noise ratio) of 3:1, limits of detection (LOD) of <10 ng/mL may be achieved. For the 1 μL samples used in the MALDI-TOFMS analytical systems, about 10 ng/mL LOD equates to a total mass of about 10 pg (10⁻¹² g) on the probe, which is equivalent to about 20,000 viral particles. An example microfluidic sample processing system to implement the method disclosed above may be configured to analyze samples collected from the air or from other sources such as nasal swabs. The glycan-based capture column and other microfluidics components may be reusable. Large fluid reservoirs containing buffer, weak acids, and alcohols may be employed to provide sufficient capacity to measure 100's of samples in one channel of the system. Multiple systems may be run in parallel to process multiple samples simultaneously. Since no fragile and expensive biomolecular reagents are required, the system is cost effective.

Hot acid digestion cleaves the proteins reproducibly at aspartic acid residues creating known peptide sequences with known masses. These peptide mass distributions are characteristic of the progenitor proteins. Thus, digestion provides outstanding specificity if the proteins of interest are largely separated from background materials. Furthermore, the peptide mass distribution is directly determined by the genome, accounting for post-translational modifications. As soon as a new virus is isolated, it is rapidly sequenced. The RNA sequence of the SARS-COV-2 virus may be used to accurately predict the protein sequences with modern bioinformatics tools (ExPASy bioinformatics portal). These proteins can then be “digested” in silico using bioinformatics tools to create a theoretical peptide map. Thus, the peptides that arise from SARS-COV-2 digestion can be predicted and compared to experimental data to generate a specific MALDI TOFMS signature of the organism. Reports suggest that the predominant proteins in SARS-COV are characterized by about 46 kDa nucleocapsid protein and the 139 kDa spike proteins. Other proteins in reasonable abundance are E, M and N proteins.

Detection specificity of a target virus will require some level of background removal, particularly if the background contains other proteins. If large amounts of exogenous proteins are present, the peptide map could be dominated by non-target peptides. As previously described, affinity capture probes for the virus toxins based on glycan-decorated agarose beads may be used to readily clean up the toxins, even in large excess of background proteins, and other biomolecules. When analyzing exhaled breath for virus targets such as SARS-COV-2, other human proteins in breath may interfere with detection specificity. An affinity-based cleanup of the sample is required to ensure high specificity. Virus detection may require bead materials that provide more selective affinity compared to the glycan-decorated beads previously described. For example, dextran-based adsorbents may be used for purifying viruses, including coronaviruses, but the affinity of this resin for the target virus may not be satisfactory. As an alternative, carbohydrates may be used for viral and protein purification including target viruses such as SARS-COV and SARS-COV-2.

In some implementations, heparin, and heparan sulfate may be used as binding agents bound to resin beads. Heparin covalently linked to sepharose beads (GE Healthcare Life Sciences, Heparin Sepharose 6 Fast Flow affinity resin Product #17099801) may be used instead of glycan capture beads. This resin may enable bead-based capture affinity capture system for collecting virus particles from exhaled breath. In an example diagnostic system, exhaled breath samples may be pulled through a capture bed in a sample collection system 1000, collecting particles from the breath. The resin beads (bed) may be washed to remove any background material. The viral particles adsorbed to the beads may then be eluted off using high concentration of acid solution, such as at least one of about 12.5% acetic acid, about 5% TFA, about 5% formic acid and about 10% HCl, into the hot acid digestion chamber to generate the characteristic peptides. The peptide samples may be mixed with MALDI matrix and deposited onto as suitable substrate for MALDI-TOFMS analysis. The samples may also be deposited on a suitable substrate or disk that is precoated with MALDI matrix.

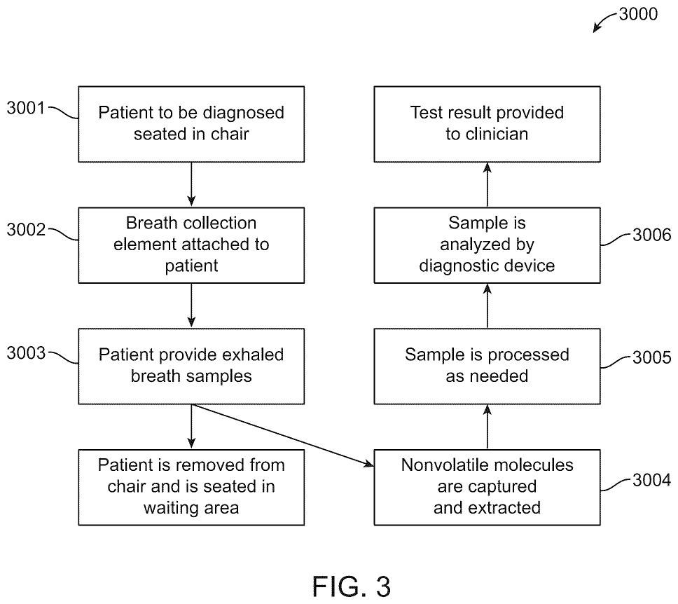

FIG. 3 shows a schematic diagram of an example diagnostic method 3000 using a system that includes a packed bed column, according to some implementations. Example method 3000 may be used to perform autonomous point-of-care diagnosis based on exhaled breath. In step 3001, the individual (or patient) may be directed to be seated and the chair may optionally be located in a containment booth. In step 3002, sample breath collection element 1007 may be removably fitted to the individual's head. The individual may then be instructed to breathe or perform one or more predetermined maneuvers 3003 which may include a pre-set number of repetitions. Non-volatile organics in breath are captured using system 1000 and extracted using system 2002 in step 3004 and eluted using suitable solvents. During sample collection, human exhaled breath passes through the column at a predetermined flow rate drawn by a pulling pump. Since nonvolatile molecules contained in the exhaled breath interact with the functionalized beads in capture element 1001 (e.g., C18 functional groups immobilized on resin beads), these molecules are trapped in the column bed in element 1001 while hydrophilic molecules including mostly of water and aqueous electrolytes in the breath pass through the column. Nonvolatile organic molecules in human breath show a strong affinity for alkyl chains via intermolecular forces including hydrogen bond and noncovalent interaction.

In some implementations, elution of nonvolatile molecules from the packed bed column may be accomplished using organic solvent that include one or more of acetonitrile, methanol, or isopropanol, as previously described. The sample may be further processed in step 3005 using component 2004. The type of sample processing depends on the type of diagnostic device and the non-volatile analyte particle of interest. As previously described, a virus sample may be subjected to hot acid digestion chamber to generate characteristic peptides. The peptide samples may be mixed with MALDI matrix and deposited onto a suitable substrate for MALDI TOFMS analysis. The samples may also be deposited on a suitable substrate or disk that is precoated with MALDI matrix. The sample may then be analyzed by a diagnostic device in step 3006. When the diagnostic device is MALDI-TOFMS, sample processing may also include the steps of plating the sample on to a MALDI-TOFMS sample disk, heating the disk to concentrate the sample, and drying the disk. The sample disk is then analyzed using a MALDI-TOFMS. The TOFMS detectors may be modified to incorporate an ion gate and a reflectron to enable analysis and sequencing of COVID-19 type virus peptides that are fragmented during MALDI-TOF/MS. The spectrum obtained is compared to spectra from samples that were known positives to specific respiratory infections, to spectra in known databases, and also to spectra of samples form patients know to be healthy, and a diagnosis of the patient is generated. The result may then be communicated to a clinician or to the patient.

In some implementations, once the breath collection element 1007 is attached to the patient, and sample extraction is initiated, the example systems and methods may be preferably autonomous (with the exception of asking the patient to the leave the chair after performing the required maneuvers) and generates a test result of the diagnosis. In the case of virus particles like SARS-COV-2, the particles are about 0.1 micron in diameter and sensitivities may be between about 10³ and 10⁴ viral particles.

Reports suggests that analysis of nose and throat swabs from influenza patients and COVID-19 patients produce viral counts of between about 10³ and 10¹⁰ viral particles. Less is known about the viral particles count in the breath of patients. Other reports suggest that influenza patients exhaled >10⁴ particles in about 30 minutes of breathing. If the output of SARS-COV-2 is similar to that of influenza, an output of 10³ to 10⁴ particles in exhaled breath with a particle collection efficiency of >99.9% should be sufficient to identify the target virus particles in exhaled breath using the example methods and systems disclosed herein. Detection time using the example systems and methods may be between about 10 minutes and 20 minutes include the steps of sample extraction (breathing maneuvers), sample collection, sample processing (digestion) and analysis using a MALDI TOF-MS. This detection time is quite rapid compared to existing detection systems. An example sample processing component may include a hot acid digestion module or cartridge to autonomously extract sample from the packed bed column 1001, perform sample clean-up, conduct the hot acid digestion and provide a sample ready for plating on a MALDI-TOFS sample substrate or disk. The cartridge may be designed for reusability by adding the capability to flush the cartridge between uses.

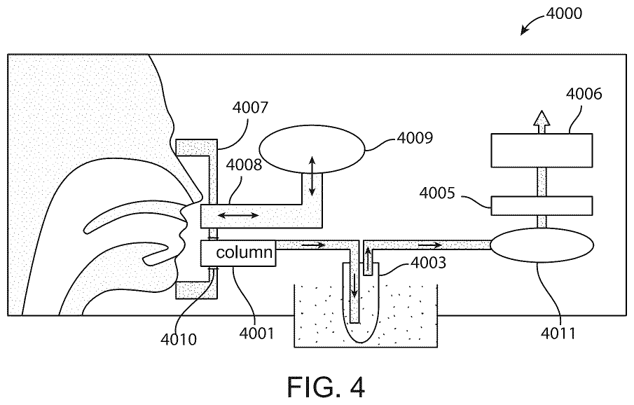

FIG. 4 shows a schematic diagram of an example exhaled breath aerosol particle capture system including a packed bed column, according to some implementations. In some implementations, the example exhaled breath capture element 4001 may include a packed bed column including C18-bonded resin beads. These resin beads may have C18 functional groups immobilized on the surface. Capture element 4001 may also be connected or removably installed to a first aid CPR rescue mask 4007 with minor modifications. In some implementations, the stem 4008 of mask 4007 that usually connects to a resuscitation bag may be modified to removably connect to a HEPA filter 4009. The HEPA filter prevents contamination of inhaled breath by contaminants from ambient air. The oxygen inlet 4010 to the mask may usually be located below the stem and may be configured to be proximate to the chin of a human subject when a mask is worn by the subject. Element 4001 may be removably inserted into mask 4007 through inlet 4010 or otherwise removably connected to or inserted into mask 4007 to form a substantially leak-tight fit with mask 4007. Mask 4007 may include elastic bands or ties that may be looped behind the head of a human subject to seal the mask to the face of the patient. Mask 4007, as described above, prevents direct contact between the mouth and the inlet of the column in element 4001, and minimizes or eliminates contamination of the column inlet by saliva and also maximizes non-volatile organic particle collection from exhaled breath.

In some implementations, trap 4003 may be immersed in ice water and may be installed after (downstream) of capture element 4001. The flow rate (air draw rate) using pump 4006 may be controlled using needle valve 4005 to pull about 600 mL/min. A nominal flow rate of between about 200 ml/min and 600 ml/min may be used. An optional HEPA filter 4011 may be installed between trap 4003 and needle valve 4005. Other fluidic components such as a check valve (see FIG. 1) may be installed in system 4000 to prevent backflow into the column bed in element 4001. CO₂ in exhaled breath passes through the column bed in element 4001. To determine if exhaled breath sample volume and/or breathing maneuvers are adequate, a CO₂ sensor may be disposed between the outlet of breath capture element 4001 and trap 4003. CO₂ monitoring allows for an approximation of the proportion of exhaled air volume.

In some implementations, a particle counter may also be installed between the outlet of element 4001 and trap 4003 to detect the size and number for particles exiting the packed bed column, which may also be used to detect saturation of the bed and breakthrough of nonvolatile organic molecules from the column bed. Example system 4000 may also include a capture element 4001 bypass line (not shown) to enable standardization of breath volume prior to routing into the column bed in element 4001. A CO₂ sensor and particle counter may also be fluidly connected to the bypass line. The capacity of solid beads immobilized with functional groups in the packed bed column in capture element 4001 may be between about 0.05 mg (non-volatile organics)/mg beads and about 0.5 mg/mg. The capacity of C18-bonded resin beads in the packed bed column disposed inside example capture element may be about 0.1 mg/mg. That is, a column bed having 25 mg C18 beads may have the capacity to trap or adsorb about 2.5 mg of non-volatile organic molecules.

Besides C18 functional groups, other functional groups that show affinity to non-volatile molecules may be used as adsorbents in the column immobilized on solid phase beads such as resin beads. In some implementations, the solid phase beads may be made of polymers and particles such as resins, cellulose, silica, agarose, and hydrated Fe₃O₄ particles. Adsorbent materials may include other functional groups that include, but are not limited to, octadecyl, octyl, ethyl, cyclohexyl, phenyl, cyanopropyl, aminopropyl, 2,3-dihydroxypropoxypropyl, trimethyl-aminopropyl, carboxypropyl, benzenesulfonic acid, and propylsulfonic acid disposed on solid phase beads. Functional groups may also include at least one of ion exchange phases, polymer phases, antibodies, glycans, lipids, DNA and RNA.

The example system and methods described herein are not necessarily limited in their diagnostic capability to respiratory infections. Lung cancer, for example, may also release biomarkers into the peripheral lung fluid, and these biomarkers would be readily detected by the systems and methods disclosed. Furthermore, because blood comes into intimate contact with the alveolar lining in the lungs, biomarkers of infection and cancer in other parts of the body (beyond the lungs) may be transferred across the alveolar lining and into the peripheral lung fluid, and thus, may be detected by the analysis of EBA. As a result, the scope of the invention is not limited to the detection and diagnosis of respiratory disease. The example systems and methods may be used to capture aerosol chemical particles such a ricin and analyze the particles to prevent a chemical attack threat.

In some implementations, the packed bed column length (L) in sample capture element 1001 may be about 3 mm. The nominal internal diameter (D) of the tube may be about 7 mm. An example packed bed including about 25 mg of C18 resin beads having a nominal particle diameter (Dₚ) of between about 12 μm and 20 μm, yields a L/Dₚ ratio of between about 150 and 250 at a D/Dₚ ratio of about 350 to about 580. These column parameters were found to prevent undesirable localized flow distributions in the bed to ensure that substantially all resin beads were exposed to the aerosol flow through the bed.

In some implementations, an exhaled breath capture module that minimizes any back pressure may be desired. Backpressure would cause extreme discomfort to the person or patient during exhalation of breath for diagnosis of a respiratory disease, in particular to patients with pulmonary problems. Accordingly, breath collection systems that include baffles or other impediments to the flow of exhaled breath are undesirable and ineffective as they would cause increased backpressure and cause extreme discomfort to the person or patient. Additionally, a capture efficiency of non-volatile organic particles aerosolized in exhaled breath of greater than 99% may be needed. Accordingly, capture of aerosolized particles using a film disposed on a porous support material is ineffective as they would be quickly saturated with moisture, water droplets, and the like in exhaled breath, leading to poor particle capture efficiency. In some implementations, an exhaled breath capture module that minimizes backpressure during capture of non-volatile organic particles aerosolized in exhaled breath over a period of at least 10 minutes, and at least 99% particle capture efficiency may be desired.

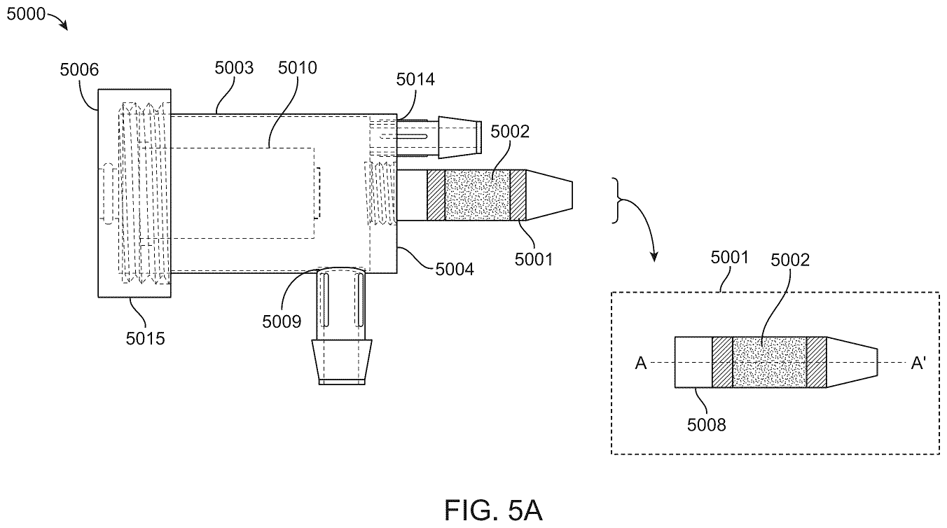

FIGS. 5A-B show schematic diagrams of an example exhaled breath capture module 5000 including a packed bed column, according to some implementations. Example breath capture module 5000 may be used to minimize backpressure during the capture of non-volatile organic particles aerosolized in exhaled breath over a period of at least 10 minutes and at least 99% particle capture efficiency. Module 5000 may include an exhaled breath capture element 5001 including a packed bed column 5002 disposed between an inlet end and an outlet end of the breath capture element. The exhaled breath capture element 5001 may selectively capture non-volatile organic particles in breath exhaled by a person. Module 5000 may include an exhaled breath management chamber 5003. In some implementations, exhaled breath capture element 5001 may be removably connected to chamber outlet end 5004. An exhaled breath tubing 5005 may be removably inserted through chamber inlet end 5006, until a gap 5007 of predetermined length is defined between an outlet end of the exhaled breath tubing 5005 and the capture element inlet end 5008. A pressure relief port 5009 may be disposed near chamber outlet end 5004, and proximate to the gap 5007. In some implementations, chamber 5003 may include more than one pressure relief port. Breath exhaled by a person may be drawn into the chamber through the exhaled air tubing using a pump (described below) disposed in fluid communication with the exhaled breath sample capture element 5001. In some implementations, breath capture element outlet end 5013 may be disposed external to the exhaled breath management chamber. In some other implementation, chamber 5003 may be configured to house the exhaled breath sample capture element 5001. In some other implementations, sample capture element 5001 may be disposed in a suitable enclosure (not shown) that abuts outlet end 5004 of chamber 5003. The pump may draw exhaled breath through the sample capture element at a nominal flow rate of between about 0.5 L/min and about 10 L/min.

In some implementations, exhaled breath management chamber 5003 may include an adapter element 5010 disposed inside the chamber to define an annular region 5011 between the adapter and the chamber. Exhaled air tubing 5005 may be removably inserted through a port disposed in the chamber inlet end 5006 and through the adapter 5010. An O-ring 5016 disposed on chamber inlet end 5006 may be used to provide a seal between tubing 5005 and chamber 5003. The adapter 5010 may include a recess 5012 to stop the extent to which the exhaled air tubing 5005 may be inserted or may travel into the adapter 5010. Recess 5012 may be positioned to define the gap 5007 of predetermined length between the outlet end of the tubing and capture element inlet 5008. That is, the recess may serve as a back-stop element in the adapter to stop the travel of the exhaled air tubing through the adapter 5010 and into the chamber 5003. The adapter 5010 may be removably disposed inside chamber 5010. The inlet end of chamber 5006 may include or may be defined by a removable chamber cap 5015. The adapter 5010 may be removed from chamber 5003 by opening the cap.

In some implementations, the non-volatile organic particles in exhaled breath to be captured by the sample capture element 5001 may include one or more of metabolite biomarkers, lipid biomarkers, proteomic biomarkers, bacteria particles, or virus particles characteristic of at least one respiratory disease. The example exhaled breath capture element 5001 may have a particle capture efficiency of greater than 99%.

In some implementations, the exhaled breath tubing 5005 may be made of one or more of paper, plastic or metal. The breath tubing may be rigid, flexible, or substantially rigid. The pressure relief port 5009 may be disposed orthogonal to a longitudinal axis (A-A′) of the exhaled breath sample capture element 5001 when the capture element is removably connected to the chamber outlet end 5004. In some implementations, the exhaled breath capture module 5000 may further include one or more instrument ports 5014 disposed at the chamber outlet end 5004. The instrument port 5014 may be disposed in fluid communication with one or more of a particle counter or a CO₂ sensor (as described below).

In some implementations, the exhaled breath capture element inlet end 5008 may have nominal diameter substantially equal to the diameter of the exhaled breath tubing. The example packed bed column 5002 may include solid particles of one or more of resins, cellulose, silica, agarose, or hydrated Fe₃O₄ particles. In some implementations, the packed bed column may include one or more of resin beads having C18 functional groups on the surface, or cellulose beads having sulfate ester functional groups on the surface. In some implementations, the resin beads or cellulose beads may have an average diameter of between about 10 μm and about 10 mm. The resin beads or cellulose beads may be packed between two porous polymeric frit discs. Example porous frit discs may include polyethylene discs supplied by Boca Scientific (Dedham, MA).

In some implementations, the beads in packed bed column 5002 may be functionalized with one or more functional groups including immobilized on the surface of the particles, wherein the functional groups include at least one of C18 (octadecyl), octyl, ethyl, cyclohexyl, phenyl, cyanopropyl, aminopropyl, 2,3-dihydroxypropoxypropyl, trimethyl-aminopropyl, carboxypropyl, benzenesulfonic acid, propylsulfonic acid, an ion exchange phase, a polymer phase, antibodies, glycans, lipids, DNA and RNA. The ion exchange phase may include one or more of diethylaminoethyl cellulose, QAE Sephadex, Q sepharose, or carboxymethyl cellulose. The polymer phase may include one or more of polystyrene-co-1,4-divinylbenzene, methacrylates, polyvinyl alcohol, starch, or agarose. The antibodies may include one or more of anti-human albumin, anti-Influenza A virus nucleoprotein (“NP”), or Anti-SARS-COV-2 virus NP.

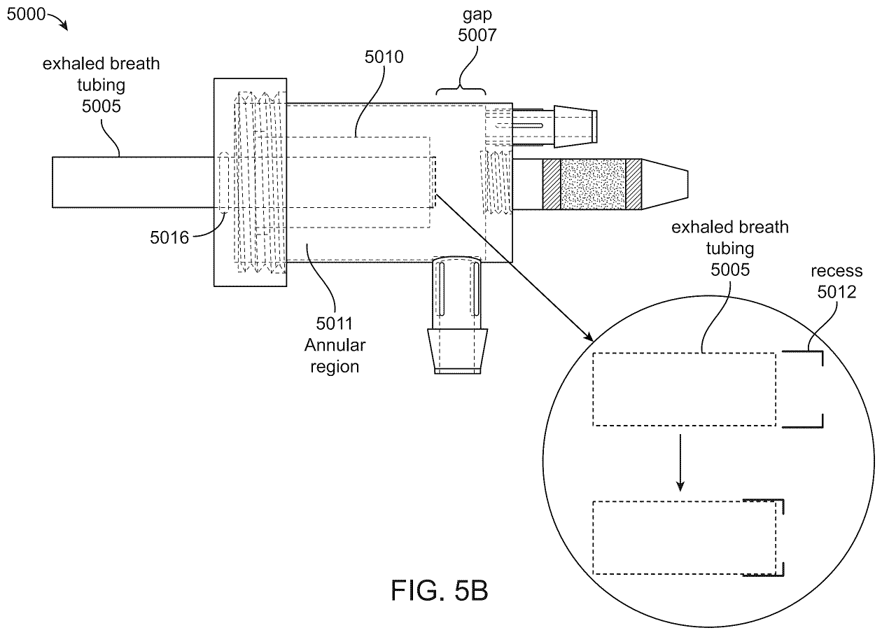

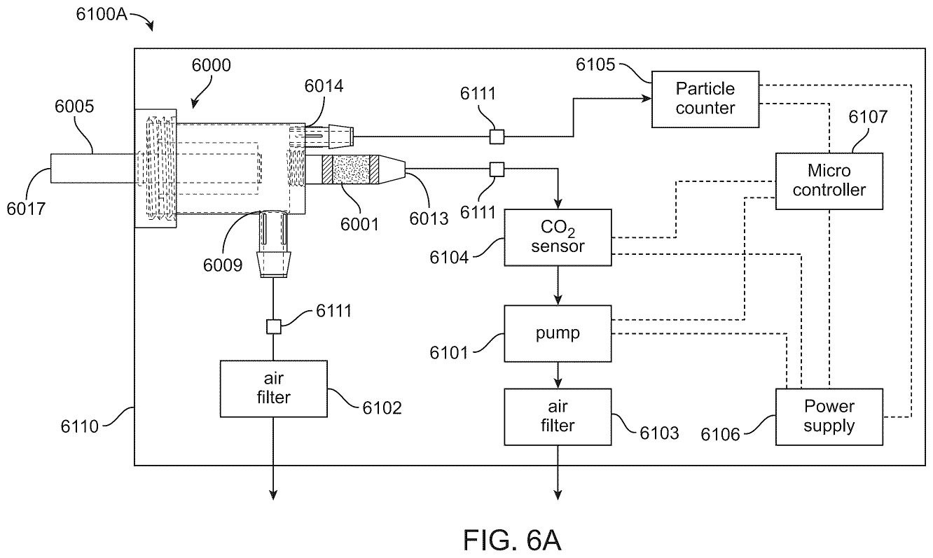

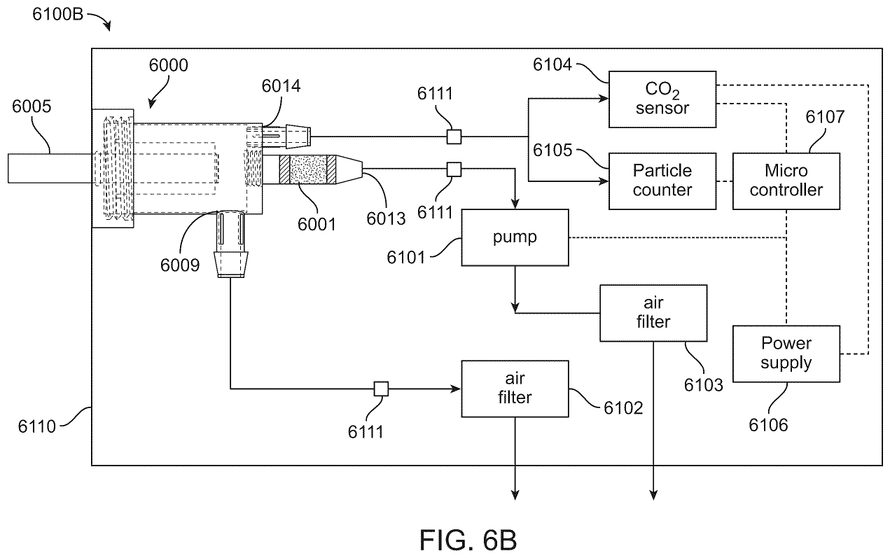

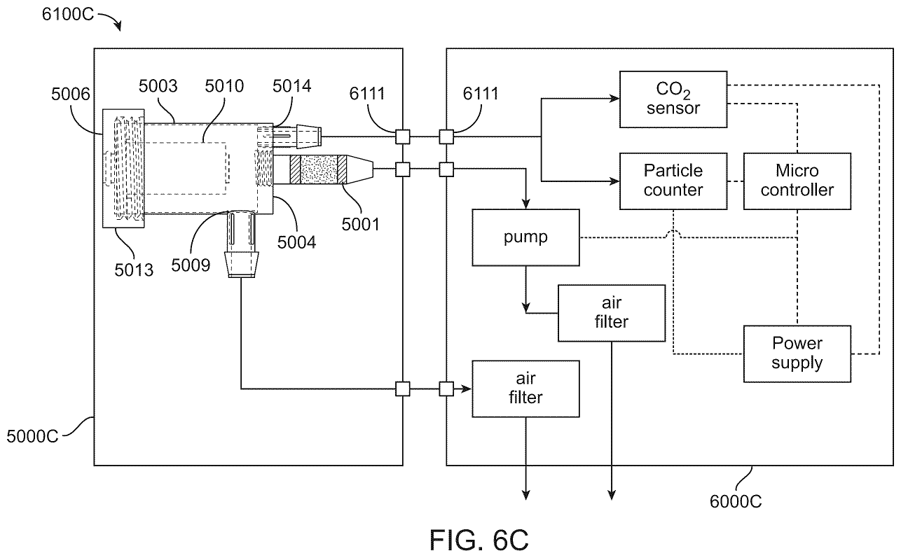

FIG. 6A shows a schematic diagram of an example exhaled breath aerosol particle capture system 6100A, according to some implementations. Exhaled breath aerosol particle capture system 6100A may include an example exhaled breath capture module 6000. Exhaled breath capture module 6000 may include the features of example exhaled breath capture module 5000, as previously disclosed herein. Exhaled breath aerosol particle capture system 6100A may include a suitable enclosure 6110. Enclosure 6110A may be configured to be opened for easy replacement of sample capture element 6001, air filters (as described below) and other components. Exhaled breath tubing 6005 may be removably inserted at one end into module 6000 for capture of aerosolized non-volatile organic particles in the exhaled breath of a person/patient. The opposite end 6017 of exhaled breath tubing 6005 may be inserted into the mouth of the person/patient. After the capture of aerosolized non-volatile organic particles in the exhaled breath according to predetermined testing protocols, tubing 6005 may be disposed. Exhaled breath capture element 6001 may be removed from system 6100 for extracting the non-volatile organic particles, as described below. Exhaled breath capture module 6000 may be replaced or removed, disinfected and reused. Module 6000 may be removably connected to pump 6101 to draw exhaled breath through the sample capture element 6001. Pump 6101 may include a diaphragm pump. A suitable fitting or coupling 6111 may be used to connect the outlet 6013 of the exhaled breath capture element 6001 to pump 6101. Fittings may be quick connect/disconnect couplings. Pump 6101 may draw exhaled breath through the sample capture element at a nominal flow rate of between about 0.5 L/min and about 10 L/min.

System 6100A may also include an air filter 6102, which may be removably connected to pressure relief port 6009 of breath capture module 6000 using a quick connect/disconnect coupling 6111. Air filter 6102 may include a HEPA filter. Exhaled breath exiting the air filter 6102 outlet may be exhausted out to the surroundings. Similarly, exhaled breath exiting the pump 6101 may be exhausted out to the surroundings after passing through another air filter 6103. One or more of a CO₂ sensor 6104 or a particle counter 6105 may be disposed in fluid communication with the instrument port 6014 of the example exhaled breath capture device 6000. In some implementations, the CO₂ sensor and particle counter may be disposed in series to each other. In some other implementations, CO₂ sensor 6104 may be disposed in series and upstream of particle counter 6105. In some implementations, the CO₂ sensor may be disposed upstream of pump 6101 as shown in FIG. 6. The CO₂ 6104 sensor may be used to determine if exhaled breath sample volume is adequate or effective for diagnostic purposes or whether additional breath maneuvers need to be implemented for a patient. CO₂ monitoring allows for an approximation of the exhaled air volume. An example CO₂ sensor may include the ExplorIR®-W CO2 Sensor (Gas Sensing Solutions), which used solid-state non-dispersive infrared (NDIR) absorption technology to measure CO₂ in concentrations of up to about 20%.

An example laser particle counter is the DR-528 particle counter (Met One Instruments, Grants Pass, Oregon) that has an internal pump to draw in exhaled breath to the particle counter. Other particle counters may be used. The particle counter 6105 may detect the size and number for particles exiting the breath capture element 6001, which may also be used to detect saturation of the bed and breakthrough of nonvolatile organic molecules from the packed bed column. Particle count and particle size data may be used to calculate particle capture efficiency of element 6001 as a function of particle size.

In some implementations, the capacity of the C18 functionalized beads in element 6001 to capture non-volatile organic particles (or molecules) may be between about 0.05 mg (non-volatile organics)/mg beads and about 0.5 mg/mg. Example C18 functionalized beads may include Hamilton PRP-C18 resin beads as supplied by Sigma Aldrich and other vendors. The capacity of C18-functionalized resin beads may be about 0.1 mg/mg. That is, a packed bed column in capture element 6001 having 25 mg C18 beads may be expected to have a capacity to trap or adsorb about 2.5 mg of non-volatile organic molecules. The C18 resin beads provide a surface affinity between the carbon chains on the beads and the organic molecules present on the surface of viruses and microorganisms. For example, the cell surface of bacteria is composed of various structures of glycans; further, wax-like mycolic acids coat the surface of M. tuberculosis. These organic molecules are generally hydrophobic and show a significant affinity to alkyl chains of the C18 beads in the packed bed column. For capturing aerosolized virus particles and virus particles in solution, example sample capture element 6001 may include sulfate ester-immobilized cellulose beads. Example sulfate beads may include Cellufine Sulfate beads (JKC Corp., Japan). As described below in Example 4, hot digestion of SARS-COV-2 virus particles extracted using a packed bed column of sulfate ester-immobilized beads, analysis using MALDI TOFMS and processing spectra using peptide fingerprint protocols may enable peptide fingerprint matching for nucleoprotein (N) and for rapid identification of SARS-COV-2.

Exhaled breath aerosol particle capture system 6100A may include a power supply 6106 to supply power to one or more of particle counter 6104, CO₂ sensor 6105, and microcontroller 6107. Data from system 6100A including one or more of data from CO2 sensor 6104, data from particle counter 6105, identifiers and personal data related to the person breathing through exhaled air tubing 6005, identifiers related to capture element 6001, identifiers related to the one or more air filters, or other operational data related to system 6110 may be transferred to a laptop or other data storage/servers for data analysis using Wi-Fi, Bluetooth or other suitable communication protocols. Data from system 6110, including the CO₂ sensor, and the particle counter may be collected and monitored on a laptop, smart-device, or other end-devices using suitable data acquisition protocols and/or interfaces. For example, an Adafruit 1141 datalogger compatible with an Arduino microcontroller may be used. Power supply may include electrical and electronic components to supply AC or DC power to the various components in system 6100A. System 6100A may further include a saliva/breath condensate cup disposed upstream of the air filter. The power and communication connections between microcontroller 6107, the power supply 6106, pump 6101, and CO₂ sensor 6104 as shown in FIG. 6 are example schematic drawings and other implementations of these connections may be used.

In some implementations, system 6100A may include a battery, which may include a primary battery or a rechargeable battery to supply power. In some implementations, system 6100A may include a graphical user interface (GUI) disposed in communication with the microcontroller 6107. Data may be recorded on a non-volatile memory card such as an SD card. A flow rate sensor may be installed to monitor the flow rate through the capture element 6001. Alternately, one or more of a flow controller or flow restrictor may be employed to achieve a consistent flow rate through the packed bed column. System 6100A may include an on-off switch to initiate and stop the capture of exhaled breath.

FIG. 6B shows a schematic diagram of an example exhaled breath aerosol particle capture system 6100B, according to some implementations. In system 6100B, the CO₂ sensor 6104 is not disposed upstream of pump 6101. Instead, CO2 sensor is disposed in fluid communication with instrument port 6014.

FIG. 6C shows a schematic diagram of an example exhaled breath aerosol particle capture system 6100C, according to some implementations. In system 6100C, the exhaled breath capture module 5000 may be disposed in a first enclosure 5000C. In this implementation, the fluidics, sensor, and control components of system 6100A may be disposed in a second enclosure 6000C. Enclosures 5000C and 6000C may be fluidly connected through connections or couplings 6111. It can be readily understood that various other packaging arrangements of the components shown in system 6100A or configurations are within the scope of this invention.

In some implementations, an example system for diagnosing a respiratory disease, may include an example exhaled breath aerosol particle capture system as previously disclosed herein, an extraction system to extract the non-volatile organic particles from the packed bed column in the sample capture element into one or more liquid samples, and an analytical device to analyze the particles in the one or more liquid samples. The extraction system may include means to flush the packed bed column with at least one solvent and to collect the solvent including aerosol particles from the packed bed. The analytical device may include one or more of PCR, ELISA, rt-PCR, mass spectrometer (MS), MALDI-MS, ESI-MS, or MALDI-TOFMS, and LC-MS/MS.

In some implementations, the non-volatile organic particles may be extracted from the beads in capture element 6001 by placing the element in a capped tube having a suitable extraction solvent such as 70% isopropanol, shaken by hand or subjected to centrifugation to extract the captured aerosols into the solvent. Some implementations, the beads in the packed bed column may be re-used after washing the beads first with organic solvents and subsequently with inorganic solvents such as water and PBS buffer solution. A diagnostic device that enables top-down proteomics such as MALDI-TOFMS may be used as the analytic device. In top-down proteomics, intact protein ions or large protein fragments from bacteria and viruses are subjected to gas-phase fragmentation for MS analysis.

In some implementations, an example method for diagnosing respiratory disease may include providing any one of the example exhaled breath aerosol particle capture systems as previously disclosed herein, inserting an exhaled breath tubing into the exhaled breath capture module, instructing a person to exhale breath via the person's mouth into the exhaled breath tubing following a predetermined protocol, removing the exhaled breath capture element from the exhaled breath aerosol particle capture system, extracting the non-volatile organic particles from the packed bed column into one or more liquid samples by flushing the packed bed column with one or more solvents, and analyzing the aerosol particles in the one or more liquid samples to determine the presence or absence of the respiratory disease. An example breath capture module may include any one of the modules previously described herein.

In some implementations, the one or more solvents may include one or more of acetonitrile, methanol, trifluoro acetic acid (TFA), or isopropanol (IPA), the remaining being water. The one or more solvents includes one or more of between about 50 vol % and about 70 vol % acetonitrile in water, between about 50 vol % and about 70 vol % isopropanol in water, or between about 0.05 vol.-% TFA in water. The extracting step may include flushing the packed bed column using one or more of about 70 vol % ACN in water, water, or about 0.05 vol % TFA in water. In some implementations, the extracting step may include flushing the packed bed column first with about 50 vol % ACN in water and subsequently with about 70 vol % IPA in water.

In some implementations, captured non-volatile aerosol organic particles may be extracted from the breath capture element 6001 by washing (or flushing) the pack bed column in element 6001 with a solvent including at least one of 70% acetonitrile (ACN), about 50% to about 70% methanol, and about 50% to about 70% isopropyl alcohol (IPA). For example, 50% ACN flush may be used to elute metabolites and proteins in a first-stage flush followed by 70% IPA flush to elute lipids from the packed bed column. The organic solvent may be removed, if needed, from the packed bed column by lyophilization overnight to preserve the captured bioaerosol particles. The organic solvent may be also removed by incubating on a heating block at about 70° C. for about 30 minutes. Finally, the bed may be washed with about 0.05% TFA. The packed bed may be washed with water at least once prior to solvent extraction to remove water soluble inorganic contaminant particles.

In some implementations, the analytical methods for the analysis of metabolites, proteins, and lipids may include silver staining for protein profiling, protein assay for protein content, bottom-up proteomics and LC-MS/MS for metabolomics and lipid-omics, and MALDI-TOF mass spectrometry for molecule profiling. In an example test, exhaled breath aerosol from patients infected with pneumonia were collected using example capture element 6001. During subsequent analysis, protein content measured using protein assay and molecule profiling measured using MALDI-TOF MS were found to be good indicators of pneumonia infection in patients as revealed by Pearson's correlation heatmap including the variables of collected total exhaled air volume, CO₂ content in exhaled air, protein content, MALDI-TOF total ion intensity and MALDI-TOF MS single peak (4820 m/z) intensity.

In some implementations, an example method for diagnosing respiratory disease may further include a sample processing step. In some implementations, the sample processing step may include mixing the one or more samples with a MALDI matrix and applying the one or more mixed samples to one or more sample plates. In some implementations, the sample processing step may include subjecting the one or more liquid samples to protein digestion and generating a peptide sample characteristic of the respiratory disease. In some implementations, the analyzing step may include analyzing the aerosol particles in the one or more liquid samples using one or more of PCR, ELISA, rt-PCR, mass spectrometer (MS), MALDI-MS, ESI-MS, MALDI-TOFMS, or LC-MS/MS. The samples may be subjected to one or more of the sample processing steps are previously described herein.

In some implementations, an exhaled breath capture element for capturing non-volatile organic particles in exhaled breath may include a packed bed column disposed between an inlet end and outlet end of the exhaled breath capture element. The packed bed column may selectively capture non-volatile organic particles in exhaled breath drawn through the packed bed column. The example packed bed column may include a first packed bed including silica gel beads. The first bed may be disposed proximate to the inlet end of the exhaled breath capture element. The example packed bed column further includes a second packed bed disposed downstream of the first packed bed. The second packed bed may include resin beads functionalized with C18 groups. The example packed bed column may be saturated with water and sealed by sealing the inlet end and outlet end of the exhaled breath sample capture element. In some implementations, the silica gel beads may have an average diameter of between about 20 μm and about 500 μm. The resins beads functionalized with C18 groups may have an average diameter of between about 20 μm and about 500 μm. The first packed bed and second packed bed may be packed between porous polymeric frit discs.

In some implementations, an exhaled breath capture element for capturing non-volatile organic particles in exhaled breath may include a packed bed column disposed between an inlet end and outlet end of the exhaled breath capture element. The example breath capture element may selectively capture non-volatile organic particles in exhaled breath drawn through the packed bed column. The example packed bed column may include a first packed bed including beads surface functionalized with one or more of amino or anionic bonded phases or groups. The first bed may be disposed proximate to the inlet end of the exhaled breath capture element. The example packed bed column may also include a second packed bed disposed downstream of the first packed bed. The second packed bed may include beads surface functionalized with reverse phases. The example packed bed column may be saturated with water and sealed by sealing the inlet end and outlet end of the exhaled breath sample capture element. The non-volatile organic particles in exhaled breath may include one or more of metabolite biomarkers, lipid biomarkers, proteomic biomarkers, bacteria particles, or virus particles characteristic of at least one respiratory disease. In some implementations, the first packed bed may include beads functionalized with one or more of aminopropyl (NH2), diols, cyanopropyl, sulfonic acid, carboxylic acid, or polyethyleneimine groups. In some implementations, the second packed bed may include beads functionalized with one or more of C18, C8, C4, C30, or phenyl groups.

In some implementations, the average diameter of the beads in the first packed bed may be greater than the average diameter of beads in the second packed bed. In some implementations, the average diameter of the beads in the first packed bed may be lesser than the average diameter of beads in the second packed bed. The beads in the one or more of the first packed bed or the second packed bed may include one or more of silica, resins, cellulose, silica, agarose, or hydrated Fe₃O₄ particles. The total amount of beads in the first packed bed and the second packed bed may be between about 10 mg and 5000 mg. In some implementations, beads of one or more average diameters or beads having one or more functional groups may be mixed and form the packed bed column disposed between an inlet end and outlet end of the exhaled breath capture element.

In some implementations, an exhaled breath capture element for capturing non-volatile organic particles in exhaled breath may include a packed bed column disposed between an inlet end and outlet end of the exhaled breath capture element. The example breath capture element may selectively capture non-volatile organic particles in exhaled breath drawn through the packed bed column. The example packed bed column may include one or more packed beds disposed in a stacked arrangement. Each packed bed may be supported between porous polymeric frits. Each packed bed may include one or more of beads of different average size, beads of different materials, or beads surface functionalized with different functional groups to minimize pressure drop across the packed bed (that is, to improve flow rate through the bed) and to increase particle capture efficiency.

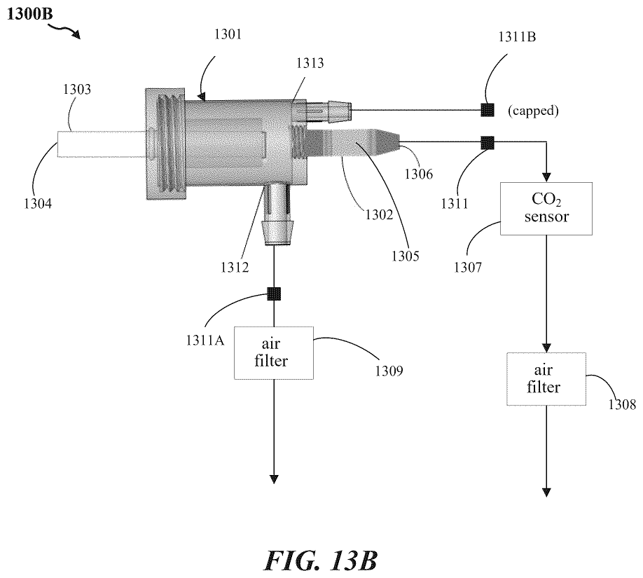

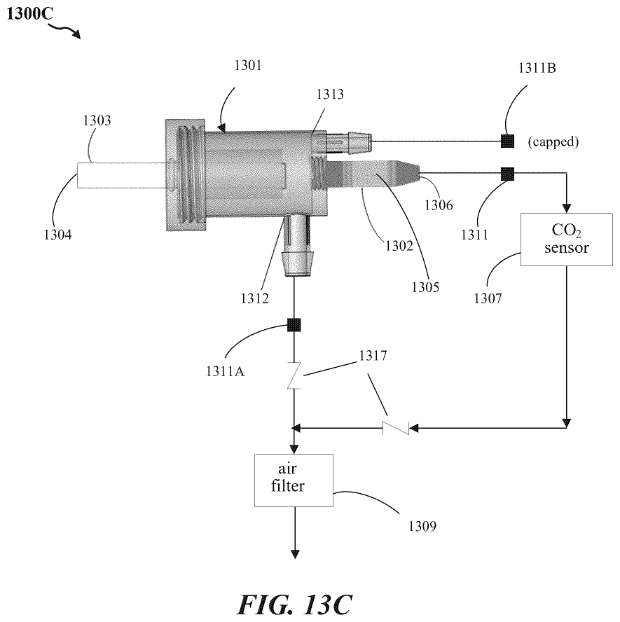

FIG. 13A shows a schematic diagram of another example exhaled breath aerosol particle capture system 1300A, according to some implementations. Exhaled breath aerosol particle capture system 1300A may include exhaled breath capture module 1301. Exhaled breath capture module 1301 may include the features of example exhaled breath capture module 5000, as previously disclosed herein with reference to FIGS. 5A-5B. Exhaled breath aerosol particle capture system 1300A may include a suitable enclosure 1310. Enclosure 1310 may be configured to be opened for easy replacement of sample capture element 1302, air filters (as described below) and other components.

In some implementations, packed bed column 1305 disposed in example exhaled breath capture element 1302 may be configured to minimize the pressure drop between the inlet end (not shown for simplicity) and the outlet end 1306 of exhaled breath sample capture element 1302. In some instances, the air flow rate through the exhaled breath sample capture element 1302 may be maximized (that is, the pressure drop across the exhaled breath sample capture element 1302 may be minimized) using one or more of a predetermined average particle size of the beads in the packed bed column, a predetermined amount of beads in the packed bed column, a predetermined length of the packed bed column, or a predetermined diameter of the packed bed column.

In some implementations, an example packed bed column 1305 may be wetted with water prior to sample capture. That is, an example packed bed column 1305 may be kept in a moist or wet state prior to use. In some instances, an example packed bed column 1305 may be washed once with 70% ACN and thrice with 0.05% TFA. In some instances, both ends of the example packed bed column 1305 may be preferably capped and stored at about 4° C. to avoid freezing. In some other instances, an example packed bed column 1305 may be capped at both ends and stored in a refrigerator to prevent drying out of the beads prior to use.

In some implementations, one end of exhaled breath tubing 1303 may be removably inserted into exhaled breath capture module 1301 for capturing aerosolized non-volatile organic particles in the exhaled breath of a person, individual, or patient. An opposite end 1304 of exhaled breath tubing 1303 may be inserted into the mouth of the person, individual, or patient. After capturing aerosolized non-volatile organic particles in the exhaled breath according to a predetermined testing protocol, exhaled breath tubing 1303 may be safely disposed. Exhaled breath may be collected for a period of about 10 min. using predetermined protocols. Exhaled breath capture element 1302 may be removed from exhaled breath capture module 1301 and from enclosure 1310 for extracting the non-volatile organic particles, as previously described herein. Exhaled breath capture module 1301 may be replaced or removed, disinfected and reused.

In contrast to the exhaled breath aerosol particle capture system 6100A (referring to FIG. 6A), exhaled breath aerosol particle capture system 1300A may not include a pump disposed downstream of exhaled breath capture element 1302 to draw exhaled breath into the exhaled breath sample capture element 1302. Instead, exhaled breath exiting exhaled breath tubing 1303 into exhaled breath capture module 1301 may flow through exhaled breath capture element 1302 unaided by a pump because of the low pressure drop across exhaled breath sample capture element 1302, as previously described herein. The elimination of the pump in exhaled breath aerosol particle capture system 1300A may reduce parasitic power draw associated with the pump and would simplify the construction or assembly of exhaled breath aerosol particle capture system 1300A without sacrificing exhaled breath aerosol particle capture efficiency. In some instances, an example particle capture efficiency associated with exhaled breath aerosol particle capture system 1300A may be at least 99%. In some other instances, an example particle capture efficiency associated with exhaled breath aerosol particle capture system 1300A may be at least 90%.

In some instances, a suitable fitting or coupling 1311 may be used to connect the outlet 1306 of the exhaled breath capture element 1302 to CO₂ sensor 1307. Example fittings may include quick connect/disconnect couplings. Exhaled breath exiting CO₂ sensor 1307 may pass through air filter 1308 to trap any exhaled breath aerosol particles before venting to the surroundings. Air filter 1308 may include a HEPA filter.

In some implementations, CO₂ sensor 1307 may include a colorimetric sensor that may be configured to change color when a predetermined volume of CO₂ is detected by CO₂ sensor 1307 when an individual breathes into exhaled breath capture element 1302 for a predetermined time. In some instances, the predetermined time may be about 10 min. Accordingly, CO₂ sensor may be a passive device that does not require electrical power for its operation. A change in color in CO₂ sensor 1307 may confirm that an effective volume of exhaled breath has passed through low-pressure drop packed bed column 1305 aided by the pressure of exhaled breath generated by the individual exhaling through exhaled breath tubing 1303. An example colorimetric CO₂ sensor may include the Nellcor™ sensor or Easy Cap II™ sold by Medtronic (Minneapolis, MN) or Covidien, or other similar example sensors. The example CO₂ sensors may change color from purple to yellow when CO₂ is detected. The example Nellcor™ sensor may have an internal volume of about 25 cc and a resistance to flow of between about 1 cm water column and about 3 cm water column; that is, between about 0.98 mbar and about 2.9 mbar. The CO₂ sensor 1307 may be used to determine if exhaled breath sample volume is adequate or effective for analytical purposes or whether additional breath maneuvers need to be implemented for a patient, individual, or person breathing out through exhaled breath tubing 1303. CO₂ monitoring allows for an approximation of the exhaled air volume.

In some implementations, exhaled breath aerosol particle capture system 1300A, may include an air filter 1309, which may be removably connected to pressure relief port 1312 of exhaled breath capture module 1301 using a quick connect/disconnect coupling 1311A. Air filter 1309 may include a HEPA filter. Exhaled breath exiting the air filter 1309 may be exhausted or vented out to the surroundings. In some implementations, the exhaled breath capture module 1301 may be capped or dead-ended at coupling 1311A.

In some implementations, a particle counter 1314 may be disposed in fluid communication with the instrument port 1313 of the example exhaled breath capture module 1301. In some implementations, the CO₂ sensor 1307 and particle counter may be fluidly connected in series to each other. In some other implementations, CO₂ sensor 1307 may be disposed in series and upstream of particle counter 1314. In some instances, particle counter 1314 may be removably connected to instrument port 1313 of exhaled breath capture module 1301 using a quick connect/disconnect coupling 1311B.

In some implementations, an example particle counter 1314 may include laser particle counter DR-528 particle counter (Met One Instruments, Grants Pass, Oregon) that has an internal pump to draw in exhaled breath to the particle counter. Other similar example particle counters may be used. The particle counter 1314 may be used to estimate the size and number for particles exiting exhaled breath tubing 1303. In some instances, when disposed downstream of exhaled breath capture element 1302, particle counter 1314 may be used to estimate the size and number for particles exiting exhaled breath capture element 1302. Accordingly, in some instances, particle counter 1314 may be used to detect saturation of the bed and breakthrough of nonvolatile organic molecules from the packed bed column 1305. Particle count and particle size data may be used to calculate particle capture efficiency of exhaled breath aerosol particle capture system 1300A.

In some implementations, exhaled breath aerosol particle capture system 1300A may include a power supply 1315 to power particle counter 1314, and microcontroller 1316. Microcontroller 1316 may include one or processors and one or more memory devices storing machine-readable code that when executed by the one or more processors may implement one or more breath collection protocols or operations and/or data collection protocols or operations. Data collected from exhaled breath aerosol particle capture system 1300A may include data from one or more of particle counter 1314, identifiers and personal data related to the person or individual breathing through exhaled air tubing 1303, identifiers related to exhaled breath capture element 1302, identifiers related to the one or more air filters, or other operational data related to exhaled breath aerosol particle capture system 1300A may be t transmitted to a laptop or other data storage/servers for data analysis using Wi-Fi, Bluetooth or other suitable communication protocols. In some instances, data from exhaled breath aerosol particle capture system 1300A may be collected and monitored on a laptop, smart-device, or other end-devices using suitable data acquisition protocols and/or interfaces. In some instances, an Adafruit 1141 datalogger compatible with an Arduino microcontroller may be used. Power supply may include electrical and electronic components to supply AC or DC power to the various components in system 1300A. System 1300A may further include a saliva/breath condensate cup (not shown for simplicity) disposed upstream of the air filter 1309. The electrical power and communication connections between microcontroller 1316, the power supply 1315, and particle counter 1314 as shown in FIG. 13A are example schematic drawings and other implementations of these connections may be implemented by those skilled in the art.

In some implementations, system 1300A may include a battery, which may include a primary battery or a rechargeable battery to supply power. In some implementations, system 1300A may include a graphical user interface (GUI) disposed in communication with the microcontroller 1316. Data may be recorded on a non-volatile memory card such as an SD card. In some instances, a flow rate sensor (not shown for simplicity) may be installed to monitor the flow rate through the capture element 1302. Alternately, one or more of a flow controller or flow restrictor may be employed to achieve a consistent flow rate through the packed bed column 1305. System 1300A may include an on-off switch (not shown for simplicity) to initiate and stop the capture of exhaled breath and the operation of on/off switch may be implemented using the microcontroller.

In some implementations, an exhaled breath aerosol particle capture system may be a passive system and may be configured to capture exhaled breath aerosol particles and may operate without any electrical power requirement. For the sake of clarity, a passive exhaled breath aerosol particle capture system may be configured to operate or otherwise collect exhaled breath aerosol particles without electrical power. FIG. 13B shows a schematic diagram of a passive exhaled breath aerosol particle capture system 1300B, according to some implementations. In contrast to the exhaled breath aerosol particle capture system 1300A (referring to FIG. 13A), system 1300B may not include particle counter 1314, power supply 1315, or microcontroller 1316. Accordingly, exhaled breath aerosol particle capture system 1300B may capture exhaled breath aerosol particles without any electrical power requirement. CO₂ sensor 1307 may be a passive device that does not require electrical power for its operation, as previously described with reference to FIG. 13A. In some instances, exhaled breath capture module 1301 may be capped (or dead-ended) at coupling 1311B or at instrument port 1313. In some other instances, exhaled breath capture module 1301 may be simplified by eliminating instrument port 1313.

In some implementations, exhaled breath aerosol particle capture system 1300B may be further simplified to provide a low-cost option for widespread use related to the analysis of exhaled breath. FIG. 13C shows a schematic diagram of another passive exhaled breath aerosol particle capture system 1300C, according to some implementations. As can be seen, exhaled air exiting passive CO₂ sensor 1307 may be routed to air filter 1309. Cross flow between exhaled breath exiting pressure relief port 1312 and exhaled breath exiting CO₂ sensor 1307 may be prevented by incorporating one or more low-crack pressure one-way valves or check valves 1317 as shown in FIG. 13C. Accordingly, exhaled breath aerosol particle capture system 1300C represents another implementation of a passive exhaled breath aerosol particle capture system or device, which may be disposed after each use. Those skilled in the art will appreciate that the schematic diagrams associated with exhaled breath aerosol particle capture systems 1300A-1300C are shown by way of example only, and that other modifications may exist without departing from the scope and spirit of the present implementations.

As previously describe herein, packed bed column 1305 disposed in example exhaled breath capture element 1302 may be configured to minimize the pressure drop between the inlet end (not shown for simplicity) and the outlet end 1306 of exhaled breath sample capture element 1302. In some instances, the pressure drop across the breath sample capture element 1302 may be less than about 500 mbar. In some other instances, the pressure drop across the breath sample capture element 1302 may be less than about 100 mbar. In some instances, the flow rate through the breath sample capture element may be at least about 3 SLPM. In some instances, the packed bed column may include beads having an average diameter greater than about 20 μm. In some instances, the packed bed column may include beads having an average diameter of about 40 μm. In some other instances, the length of the packed bed column 1305 may be about 3 mm. In some instances, the diameter of the packed bed column may be about 10 mm. In some instances, an example amount of beads in packed bed column 1305 may be about 25 mg. The beads may be packed between polymeric frit discs. The average pore size of the upstream disc and downstream disc may be about 35 μm and about 10 μm, respectively.

In some instances, packed bed column 1305 may include one or more of resin beads having C18 functional groups on the surface, cellulose beads having sulfate ester functional groups on the surface, and mixtures thereof. As previously noted, the C18 resin beads provide a surface affinity between the carbon chains on the beads and the organic molecules present on the surface of viruses and microorganisms. For example, the cell surface of bacteria is composed of various structures of glycans; further, wax-like mycolic acids coat the surface of M. tuberculosis. These organic molecules are generally hydrophobic and show a significant affinity to alkyl chains of the C18 beads in the packed bed column. For capturing aerosolized virus particles and virus particles in solution, example sample capture element 6001 may include sulfate ester-immobilized cellulose beads. Example sulfate beads may include Cellufine Sulfate beads (JKC Corp., Japan).

In some implementations, example packed bed column 1305 may include a first packed bed including silica gel beads. The first packed bed may be disposed proximate to the inlet end of the exhaled breath capture element. The example packed bed column 1305 may further include a second packed bed disposed downstream of the first packed bed. The second packed bed may include resin beads functionalized with C18 groups. The example packed bed column may be saturated with water and sealed by sealing the inlet end and outlet end of the exhaled breath sample capture element. In some implementations, the silica gel beads may have an average diameter of at least about 20 μm. The resins beads functionalized with C18 groups may have an average diameter of at least about 20 μm. The first packed bed and second packed bed may be packed between porous polymeric frit discs.

In some implementations, example packed bed column 1305 may include a first packed bed including beads surface functionalized with one or more of amino or anionic bonded phases or groups. The first bed may be disposed proximate to the inlet end of the exhaled breath capture element. The example packed bed column may also include a second packed bed disposed downstream of the first packed bed. The second packed bed may include beads surface functionalized with reverse phases. The example packed bed column may be saturated with water and sealed by sealing the inlet end and outlet end of the exhaled breath sample capture element.

In some implementations, non-volatile organic particles in exhaled breath may include one or more of metabolite biomarkers, lipid biomarkers, proteomic biomarkers, bacteria particles, or virus particles characteristic of at least one respiratory disease. In some implementations, the first packed bed may include beads functionalized with one or more of aminopropyl (NH2), diols, cyanopropyl, sulfonic acid, carboxylic acid, or polyethyleneimine groups. In some implementations, the second packed bed may include beads functionalized with one or more of C18, C8, C4, C30, or phenyl groups.

In some implementations, the average diameter of the beads in the first packed bed may be greater than the average diameter of beads in the second packed bed. In some implementations, the average diameter of the beads in the first packed bed may be lesser than the average diameter of beads in the second packed bed. The beads in the one or more of the first packed bed or the second packed bed may include one or more of silica, resins, cellulose, silica, agarose, or hydrated Fe₃O₄ particles. In some implementations, beads of one or more average diameters or beads having one or more functional groups may be mixed to form the packed bed column disposed between an inlet end and outlet end of the exhaled breath capture element.

In some implementations, example packed bed column 1305 may include one or more packed beds disposed in a stacked arrangement. Each packed bed may be supported between porous polymeric frits. Each packed bed may include one or more of beads of different average size, beads of different materials, or beads surface functionalized with different functional groups to minimize pressure drop across the packed bed (that is, to improve flow rate through the bed) and to increase particle capture efficiency.

In some implementations, non-volatile exhaled breath aerosol (EBA) particles captured in the packed bed column 1305 may be extracted using one or more solvents to generate one or more liquid samples including the EBA particles. The liquid samples may be analyzed using one or more analytical devices including one or more of using one or more of PCR, ELISA, rt-PCR, mass spectrometer (MS), MALDI-MS, ESI-MS, MALDI-TOFMS, or LC-MS/MS.

In some implementations, an example passive exhaled breath aerosol particle capture device for capturing non-volatile organic particles in exhaled breath may include an exhaled breath tubing including a packed bed column disposed therein. In some instances, a passive CO₂ sensor may be disposed downstream of the packed bed column. Examples of passive CO₂ sensors were previously described herein. In some other instances, one or more colorimetric CO₂ sensor materials may be disposed downstream of the packed bed column in the exhaled breath tubing, which may include a window for detecting or seeing a color change. In some instances, the exhaled breath tubing may be made of one or more of plastic or metal. In some instances, the packed bed column may include solid particles or beads including one or more of resins, cellulose, silica, agarose, or hydrated Fe3O4 particles. In some other instances, the packed bed column may include cellulose beads having sulfate ester functional groups on the surface. In some instances, an example packed bed column may include resin beads having C18 functional groups on the surface. In some other instances, the beads in the packed bed column may have an average diameter of between about 40 μm and about 120 μm. The beads may be supported between suitable porous materials. Accordingly, the example passive exhaled breath aerosol particle capture device may be disposable.

In some implementations, the example packed bed column disposed in an exhaled breath tubing may be wetted with water prior to sample capture. That is, an example packed bed column may be kept in a moist or wet state prior to use. In some instances, an example packed bed column may be washed once with 70% ACN and thrice with 0.05% TFA. In some instances, both ends of the example packed bed column may be preferably capped and stored at about 4° C. to avoid freezing. In some other instances, an example packed bed column may be capped at both ends and stored in a refrigerator to prevent drying out of the beads prior to use.

### EXAMPLES

### Example 1. Capture and Analysis of Aerosolized Bacteria and Virus Particles Using an Example Packed Bed Column 700 and MALDI TOF-MS

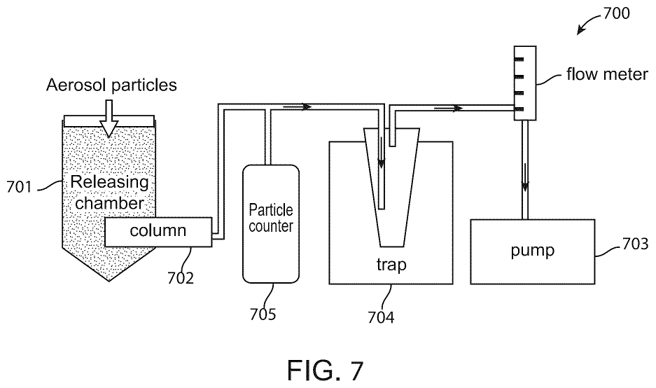

FIG. 7 shows a schematic diagram of an example aerosol sample collection test system including a packed bed column, according to some implementations. Capture and analysis of aerosolized E. coli K12 strain, Bacteriophage MS2, Pseudomonas fluorescens 1013, and Yersinia rohdei CDC 3022-85 were examined. Aerosol particles from a water sample including about 20 μL of viruses or bacteria were generated using a Sono-Tek ultrasonic nozzle (Milton, NY). The nozzle was tuned to generate aerosol particles with size ranging from about 0.3 μm to about 10 μm, with median particle size of about 2 μm. As shown in FIG. 7, the particles were directed into releasing chamber 701 (a 50 ml conical tube) having a nominal volume of about 50 ml and drawn through the packed bed column at a flow rate of about 500 ml/min for about 10 min. The packed bed column in capture element 702 included about 25 mg of C18 resin beads of about 20 μm in size. The beads were washed with 70% acetonitrile once and thrice with 0.05% trifluoroacetic acid. After washing, the beads were kept wet before use. For this purpose, column 702 may be capped at both ends and stored in a refrigerator to prevent drying out of the beads prior to use.

The column bed was about 3 mm in length and about 7 mm in diameter. The bed volume was about 0.115 cc. At a flow rate of 500 ml/min, the gas hourly space velocity (GHSV, ratio of flow rate to bed volume) was therefore calculated to be about 260,000 per hour. Capture element 702 was removably installed near the bottom of the 50 ml conical tube. A portable laser particle counter (MetOne Instruments, Grants Pass, OR) 705 was used to measure particle sizes that ranged from about 0.3 and about 10 μm upstream and downstream of capture element 702. Trap 704 included a laboratory glass reservoir cooled in ice water and was disposed downstream of element 702 to collect aerosol condensates (e.g., water vapor) passing through the column. The particle counts upstream and downstream of capture element 702 for Bacteriophage MS2 are shown in Table 1.

TABLE 1
Particle capture efficiency of example capture
element 702 for aerosolized MS2 capture.

| | Particle count | Particle count |
|---|---|---|
| Particle | measured upstream | measured downstream |
| size, μm | of capture element 702 | of element 702 |
| 0.3 | 3,921 | 12 |
| 0.5 | 4,236 | 10 |
| 1 | 15,232 | 52 |
| 2 | 17,892 | 7 |
| 5 | 1,250 | 2 |
| 10 | 365 | 0 |
| TOTAL | 42,896 | 83 |

Based on the particle counts shown in Table 1, about 99.8% of the MS2 particles were captured in the packed bed column in capture element 702 suggesting that the particle capture efficiency is at least 99% even at gas hourly space velocities of about 260,000 per hour. The packed bed column dimensions (length, diameter) and flow rate through the bed may be changed to realize gas hourly space velocities of at least 250,000 per hour. Without being bound by any particular theory, particle capture efficiency of at least 99% may be measured at gas hourly space velocities of between about 250,000 per hour and about 3,000,000 per hour. A capture efficiency of at least 99% was also measured for aerosolized E. coli particles. The example packed bed column of length of about 3 mm therefore yielded significantly higher particle capture efficiency including particles as small as about 0.3 μm and exceeded the capture efficiency using filter substrates such as electret and Teflon filters that are between about 0.05 mm and about 0.15 mm thick. As previously described, L/Dp of the packed bed is between about 150 and 250 which prevents localized flow through the bed and exposes substantially all of the beads and surface area for trapping (chemical adsorption or physical adsorption) the bio-aerosol particles. After particle capture for about 10 min., the packed bed column was washed with about 400 μL of water thrice, after which, trapped biomaterials were eluted with 200 μL of 70% isopropanol. Samples collected during the wash and elution steps were analyzed using mass spectrometry.

MALDI-TOF mass spectra of the samples were acquired using a Shimadzu Axima CFR-plus mass spectrometer operated in the linear mode from 1000 to 15000 m/z. For direct infusion and nanoflow-LC mass spectrometry, a LTQ Orbitrap system coupled with an EASY-nLC 1000 system was used (Thermo Fisher Scientific). The flow rate for direct infusion was about 3 μL/min. For LC-MS analysis, samples were injected into an microflow C18 column (Acclaim™ PepMap™ 100, 75 μm×2 μm×25 cm, Thermo Fisher Scientific) and proteins were separated using a gradient of solvent B (99% acetonitrile with 0.1% formic acid) from 5% to 65% in 90 minutes. Ion fragmentation was conducted using the collision-induced dissociation (CID) method. To improve ion fragmentation coverage, a staged-CID approach was used, and top-down mass spectra were acquired using collision energy of 0%, 10%, 15%, 20%, 25%, 30%, and 35%, respectively. During top-down mass spectrometric data analysis, monoisotopic masses were deconvoluted using Xcalibur software (Thermo Fisher Scientific) and the fragmentation ions were examined and identified using ProSight Lite (Northwestern University).

The wash samples and elution samples (after extraction of the packed column bed) were analyzed using MALDI-TOFMS. Results showed that bacterium signatures were well represented in the elution samples suggesting that the aerosol capture and elution methods and systems disclosed herein provide for analysis of the complete or original (whole cell) biological materials. Further, the signal-to-noise between the control and elution samples was indistinguishable suggesting the capture using the packed column bed and extraction was highly effective. In fact, after a quick centrifugation on the elution samples, bacterium materials were visualized as pale pellets on the bottom of the tube, suggesting an excessively strong capture capacity of the collection system.

Most viruses have a protein shell. MS2 was used as a representative virus model to evaluate the ability of the disclosed example systems and methods to capture viruses. MALDI-TOF mass spectrometric analysis showed that capsid protein, which is the historical biomarker of MS2, was observed in the elution sample. The identity of MS2 capsid protein (13729 Da, MH⁺) was confirmed using a top-down mass spectrometry approach in which high-confident statistical scores were constructed when matching the experimental fragmentation ions against in silico protein fragmentation pattern. Therefore, MALDI-TOFMS whole cell characterization and top-down protein identification were used to confirm that the disclosed aerosol capture systems and methods may be used to capture aerosolized virus particles.

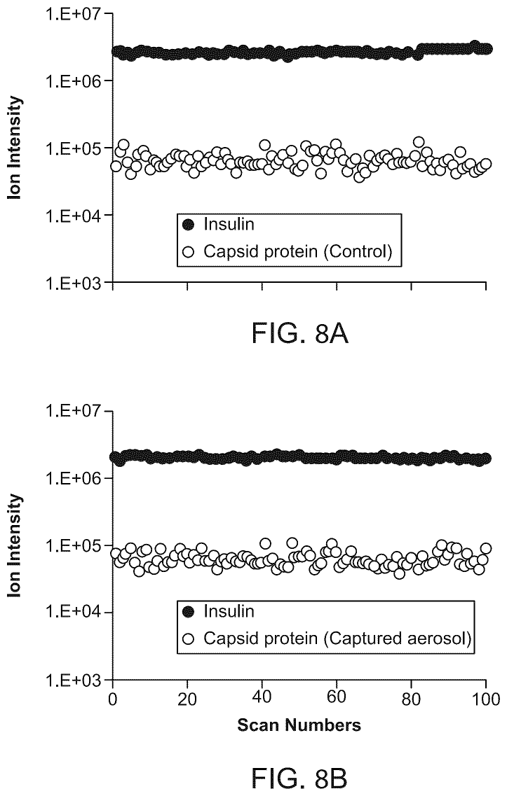

To further confirm the results observed using top-down mass spectrometry, direct infusion mass spectrometry and nanoflow LC mass spectrometry were used for quantitative analysis. FIG. 8A shows direct infusion mass spectrometry results showing ion intensities of MS2 capsid protein in the control sample (referee), according to some implementations. FIG. 8B shows direct infusion mass spectrometry results showing ion intensities of MS2 capsid protein in the captured aerosol sample, according to some implementations. Direct infusion mass spectrometric analysis showed that the signal intensity of referee insulin was about 3.6E+6. The signal intensity of MS2 capsid protein was about 1.1E+5 in both control sample and capture samples (sample obtained by extracting aerosols from the packed bed column), suggesting no obvious sample loss is observed using the example aerosol capture system and methods and MALDI-TOFMS analysis.

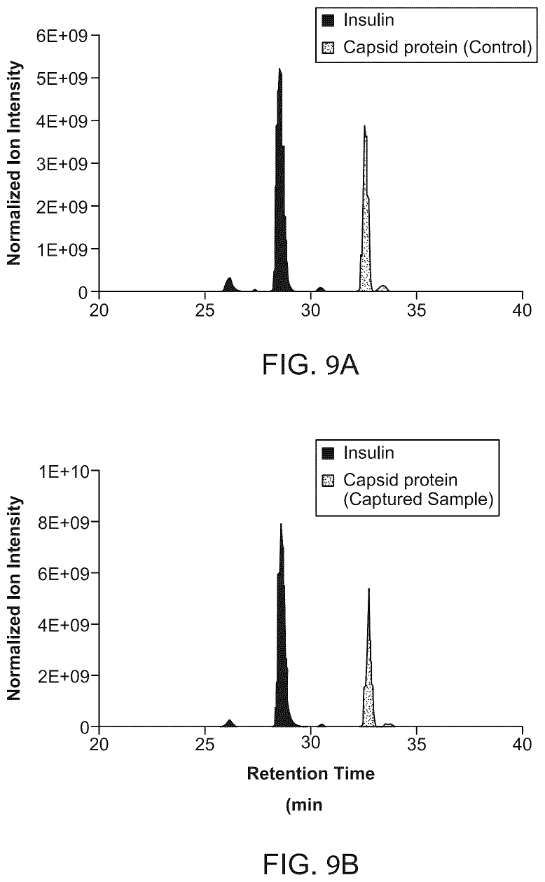

This observation was further confirmed using nano-flow LC mass spectrometry, a gold standard for molecule quantitative analysis. FIG. 9A shows nanoflow-LC mass spectrometry selected ion chromatogram of MS2 capsid protein in a control sample (referee), according to some implementations. FIG. 9B shows nanoflow-LC mass spectrometry selected ion chromatogram of MS2 capsid protein in a captured aerosol sample, according to some implementations. LC mass spectrometry results showed that the peak areas and ion intensity of MS2 capsid protein were substantially identical in both control and capture samples, suggesting the example aerosol capture systems and methods preserved a high biochemical capture efficiency.

Without being bound by any particular theory, several factors contribute to the superior capture efficiency of the disclosed aerosol capture methods even at gas hourly space velocities through the bed, including, but not limited to, the high retention capacity of C18 resin beads for aerosolized organic biomaterials such as virus and bacteria particles, the preparation of the packed bed prior to use, disposing the capture element directly to the source of aerosol particles such as aerosol releasing chamber 701, and the unique packed bed column parameters employed for capturing aerosolized particles. As previously described, the dimensions of the packed bed column provide sufficient retention time at flow rates of about 500 ml/min and ensure good contact between carbon chains on the resin beads and the organic molecules on the surface of viruses. In addition, since aerosol particles including viruses and microorganisms were directly exposed to the C18 beads “wall-losses” were minimized. Wall losses are typically present in other aerosol collection methods in which particles are deposited on the sides or walls of the device. Quantitative analysis of MS2 capsid protein demonstrated superior capture efficiency of the disclosed aerosol collection systems and methods. In addition, since no vacuum pump is used, the disclosed systems and methods may be treated as a “soft collection techniques,” because they preserve the viability of whole-cell viruses, bacteria, and other microorganisms that are required for other microbiological culture studies.

### Example 2. Example Packed Bed Column Characteristics

The use of membrane-based aerosol collection devices is plagued by clogging and pressure drop issues, which is caused by the accumulation of water droplets and other environmental particles during aerosol collection. This requires a large membrane surface area, which increases costs, and high parasitic power and noisy air pumps. Aerosol capture tests were conducted using C18 beads of nominal diameter of about 10 μm and about 20 μm. The beads were packed between polymeric frit discs. The average pore size of the upstream disc and downstream disc was about 35 μm and 10 μm respectively. HPLC-grade water and E. coli vegetative cells were aerosolized into chamber 701 and pulled through an example packed bed column 702 using pump 703, which is preferably a diaphragm pump. E. coli is a rod-shaped bacteria with nominal dimensions of between about 1 μm and about 2 μm in length and about 1 μm in diameter. The pump flow rate was set at 2.5 L/min and actual flow rate through the bed was measured using a flow meter (see FIG. 7). Tests were conducted using a packed bed column of C18 beads.

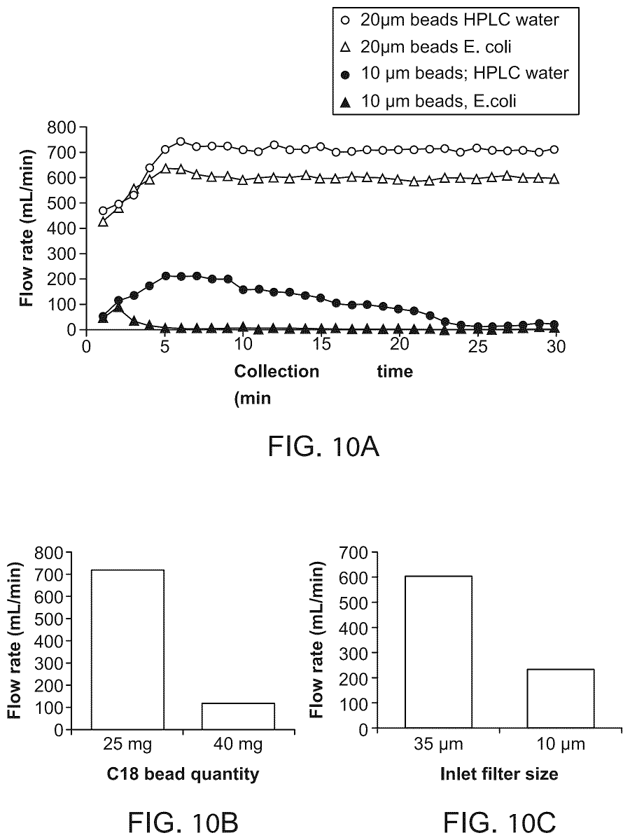

FIG. 10A shows test results showing the effect of C18 bead size on flow rate through an example sample capture element, according to some implementations. As shown in FIG. 10A, the measured flow rate through the bed including about 25 mg, 20 μm C18 beads was about 750 ml/min when the bed (about 3 mm in length) was exposed to HPLC-grade water aerosols, and about 600 ml/min when the bed was exposed to E. coli aerosols over 30 min of capture time. This result suggests that the 25 mg bed of about 20 μm, C18 beads was not saturated during these tests indicating that the amount of the beads in the bed may be reduced. Alternately, the capture time may be increased using a 25 mg bed. In contrast, when the bed included 10 μm C18 beads, flow rate decreased from about 200 ml/min to about 50 ml/min in about 30 min when the bed was exposed to HPLC-grade water aerosols. Further, flow rate decreased from about 100 ml/min to about 3 ml/min when the bed was exposed to E. coli aerosols indicating significant pressure drop through the bed including 10 μm beads. These results suggest that C18 beads in the packed bed column in capture element 702 of particle size of at least about 20 μm is preferred.

Additionally, the impact on flow rate reduction due to increasing pressure drop across the bed was examined by varying the quantity of C18 beads in the packed bed column, and pore size of the polymeric frit disc disposed at the inlet end of the bed. The pump flow rate was set at 2.5 L/min and the actual flow rate through the bed was measured using a flow meter. The amount of C18 beads of nominal diameter of about 20 μm was increased from about 25 mg to about 40 mg in the packed bed column. The average pore size of the upstream disc and downstream disc was about 35 μm and 10 μm respectively. FIG. 10B shows test results showing the effect of bead quantity on flow rate through an example sample capture element, according to some implementations. Increasing the bed amount from about 25 mg to about 40 mg increased the bed length from about 3 mm to about 5 mm. In each case, the flow rate through the bed was monitored after exposing the bed to aerosolized HPLC-grade water aerosols. The results depicted in FIG. 10B show that increasing the amount of C18 beads in the bed from about 25 mg to about 40 mg resulted in a decrease in the flow rate from about 700 ml/min to about 150 ml/min after 30 min of collection time.

Further, in a separate set of tests, the pore size of the inlet polymeric frit disc was changed from 35 μm to about 10 μm. FIG. 10C shows test results showing the effect of inlet polymeric frit pore size on flow rate through an example sample capture element, according to some implementations. During exposure to aerosols of E. coli (vegetative cells), the flow rate decreased from about 600 ml/min to about 250 ml/min during the 30 min. collection time.

### Example 3. Aerosolized Protein Capture Using Example Sample Capture Element with C18 Packed Bed Column and a Commercial Button Sampler

The example sample capture element included a packed bed column of C18 beads of nominal diameter between about 45 μm and 120 μm. The amount of C18 beads in the column was about 200 mg and bed length was about 3 mm. The length and diameter (ID) of the capture element was about 60 mm and about 15.9 mm respectively. The Button Sampler (SKC Ltd, United Kingdom) included a filter sampler (25 mm filters with pore size greater than 1 μm) with a porous curved-surface inlet designed to improve the collection characteristics of inhalable dust (100 μm aerodynamic diameter), including bioaerosols for total microbial count.

Protein solution including BSA protein in artificial saliva (NCZ-APS-0012) background solution with a BSA concentration of about 2 mg/ml was aerosolized using a Dynamic Concentration Aerosol Generator at the Applied Physics Laboratory at Johns Hopkins University. In the case of the example sample capture element, a mechanical pump was used to draw the aerosol through the packed bed column at about 10 L/min. After sample collection, the C18 beads packed bed column was eluted with 2 mL of 70% CAN and the organic solvent was removed by an overnight lyophilization. The sample was suspended in 50 μL of water and used for SDS-PAGE electrophoresis, blue staining, and bottom-up proteomics.

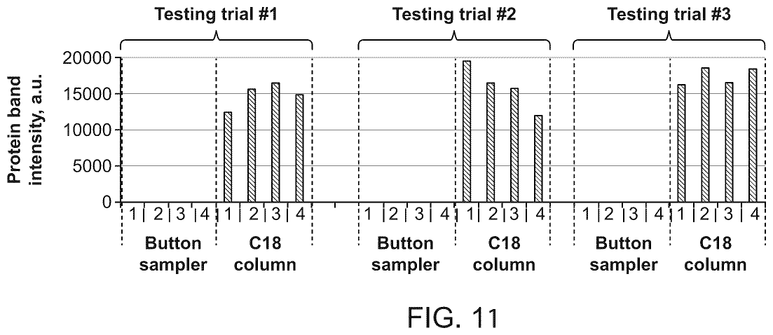

FIG. 11 shows protein blue staining results of aerosolized BSA proteins captured using an example C18 packed bed column sample capture element and a button sampler, according to some implementations. As can be seen, the protein capture efficiency of the C18 example packed bed column was significantly better than that obtained using the button sampler. Further, in the case of silver staining, BSA protein bands (about 66 kDa) were not seen in samples collected with the button sampler. In contrast, BSA protein bands were seen in all samples collected with the example C18 packed bed column. Besides intact BSA protein, its dimmer form (about 140 kDa) and trimmer form (about 200 kDa) were seen in all samples collected with the C18 packed bed column.

### Example 4. Capture of SARS-COV-2 Virus Using Sulfate Ester Functional Groups and Analysis

SARS-COV-2 South African strain was grown using the cell line Vero E6 TMPR332. 0.3 mL of the virus was combined with PBS buffer solution to make a final volume of 4.7 mL. The growth media was decanted from the tissue culture vessel and gently washed thrice with 20 mL of PBS. 5 mL of diluted virus was pipetted to a vessel and incubated fat 37° C. for 15 minutes. 20 mL of un-supplemented media was added to each vessel and incubated for 2 days until CPE of about 80% was achieved. At the time of harvest, the CPE was 90%. TCID50 was concentrated to 2.32 e+7 (calculated by Reed & Muench method). In a purification step, the virus was ultra-centrifuged at 100,000g for 60 min., the supernatant was decanted, and the pellet was resuspended in 1 mL of water. The virus was then heat inactivated at 70° C. for 30 min.

The sample capture element included sulfate ester-immobilized cellulose beads (Cellufine® Sulfate). The packed bed column included beads of diameter of between about 40 mm and about 130 mm. The packed bed column (30 mm length×6.8 mm ID) included about 100 mg of beads. The column was washed once with 1 mL of washing solution (0.2 M NaCl in 1×PBS). 900 μL of the SAR-COV-2 virus sample was prepared in 100 μL of 10×PBS to make a final solution of 1×PBS solution. The entire sample solution with the virus was loaded into the column using a syringe and pushed through the column. The sample exiting the column was collected and pushed through the column 5 more times to maximize virus capture in the bed. To completely remove non-binding proteins, 2 mL of 0.2 M NaCl in 1×PBS washing solution was pushed through the column three times. Subsequently, air was pushed through the column to ensure that washing solution was left in the column. The captured virus particles were then eluted using 900 μL of 1.5 M NaCl in 1×PBS elution solution.

The eluted sample was split into two samples and each sample was subjected to hot acid digestion. Prior to this step, the eluted sample was filtered in a centrifuge. 200 μL of eluted virus was combined with 250 μL of HPLC water and loaded into a 3k (3 kDa MWCO) filter column. The sample was spun in a microcentrifuge at 14,000g for 25 minutes. The 3k column was topped off with 450 μL of HPLC water and spun again. This process was repeated thrice. The column was flipped upside-down in a new hold tube and centrifuged for 10 minutes at 10,000g. This sample was used for hot acid digestion. The sample produced from the 3k filtration was topped with about 50.5 μL of water to make a final sample volume of 87.5 μL. 12.5 μL of acetic acid was added to make a 12.5% acid solution (100 μL in total). The samples were subjected to hot acid digestion at about 140° C. for about 15 min.

The sample was then analyzed using MALDI-TOF mass spectrometry. CHCA matrix was prepared in 70% acetonitrile at a concentration of about 9 mg/mL. About 1 μl of the hot acid samples was deposited onto a MALDI plate. After the sample was partially dried, about 1 μl of the CHCA solution was deposited on top and mixed by pipetting up and down. The samples were analyzed using a commercial Bruker Daltonics microflex LRF MALDI-TOF mass spectrometer. MALDI-TOF mass spectra were obtained in the positive linear mode and an average of 600 profiles were collected in a mass range of 700-21,000m/z for all spectra. The online program MASCOT Peptide Mass Fingerprint (Matrix Science, Boston, MA) was used to identify peptide mass fingerprints. Mass peaks with S/N>10 were extracted into “Mass values” for analysis. “SwissProt” protein database was used, and “All entries” was selected for taxonomy. Enzyme was defined as “Formic_acid,” and different missed cleavage numbers, 0-9, were allowed. Peptide tolerance was set to 0.5 Da and monoisotopic type was selected.

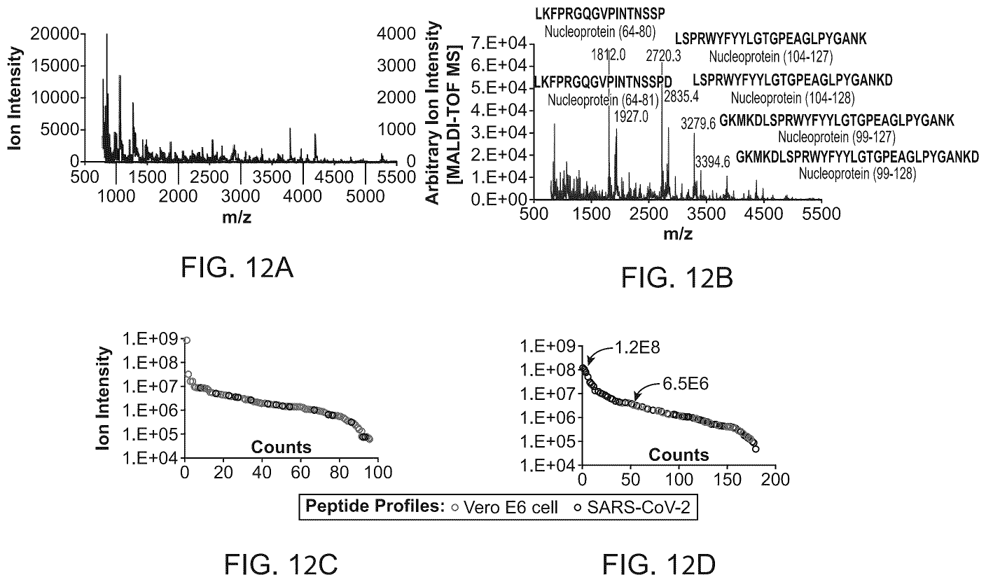

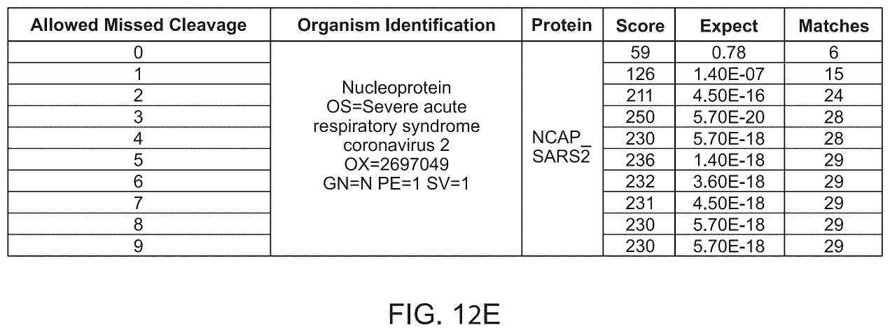

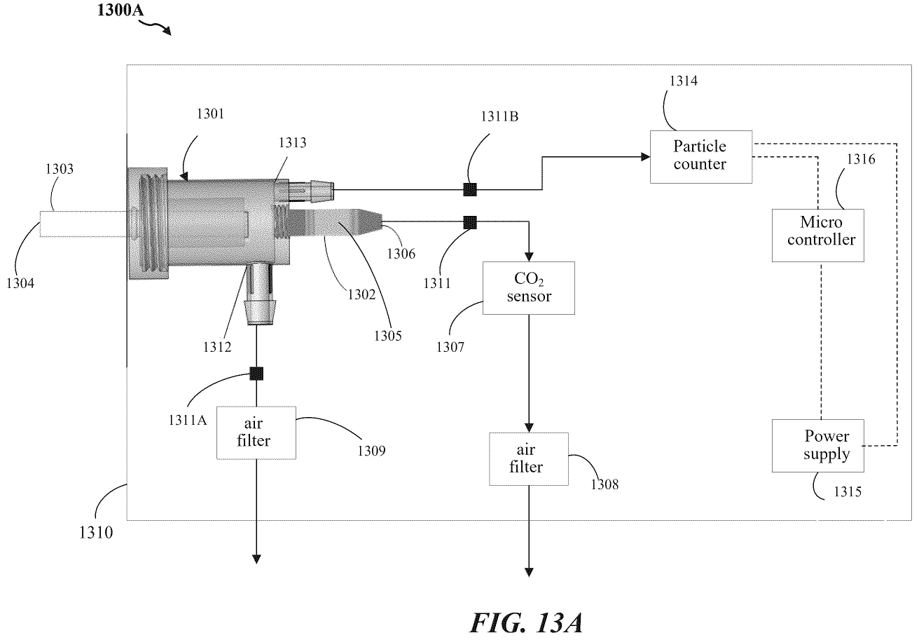

FIG. 12A shows MALDI TOF mass spectra of hot acid digested peptides in culture media of SARS-COV-2 virus sample not treated with example exhaled breath capture element, according to some implementations. FIG. 12B shows MALDI TOF mass spectra with peak assignments of hot acid digested peptides of SARS-COV-2 virus particles extracted from an example exhaled breath capture element, according to some implementations. The exhaled breath capture element included a sulfate ester packed bed column. Analysis of hot acid digested peptides of SARS-COV-2 virus particles extracted from an example exhaled breath capture element showed a more distinct profile compared to spectra of samples that were not treated with the sulfate ester packed bed column.

FIG. 12C shows distribution of peptide ion intensities constructed using bottom-up proteomics of SARS-COV-2 virus particles, according to some implementations. Bottom-up proteomics was used for peptide identification and showed that peptides of Vero E6 cell proteins were most abundant in the samples that were not treated with the sulfate ester packed bed column.

FIG. 12D shows distribution of peptide ion intensities constructed using bottom-up proteomics of SARS-COV-2 virus particles extracted from an example sample capture element, according to some implementations. In contrast to the results shown in FIG. 12C, samples extracted from the sulfate ester packed bed column showed that peptides of SARS-COV-2 proteins were most abundant. Viral peptides were also characterized by significantly higher ion intensities (FIG. 12D) in the samples extracted from the sulfate ester packed bed column. These results demonstrate that sulfate ester immobilized cellulose beads may be used for capture of aerosolized virus particles. Further, bottom-up proteomics showed that the identified peptides correspond to N protein characteristic of SARS-COV-2. Using peptide identification information from bottom-up proteomics, six mass peaks were identified in MALDI-TOF MS spectra (FIG. 12B) characteristic of SARS-COV-2. The typical mass shifting signature in hot acid-assisted hydrolysis, +115.1, was observed in these peak assignments.

Since most of the peptides identified after the enrichment correspond to N protein, MALDI-TOF MS mass peaks from samples extracted from the example capture element may be used for the identification of N protein using peptide mass fingerprinting and following SARS-COV-2 detection. FIG. 12E shows peptide mass fingerprint of SARS-COV-2 virus particles extracted from an example sample capture element using Mascot database search using different allowed missed site cleavage numbers, according to some implementations. 37 mass peaks with the signal-to-noise ratio greater than 10 were selected and processed with MASCOT Peptide Mass Fingerprint program. Since hot acid-assisted protein hydrolysis causes missed cleavage, the effect of allowed missed cleavage on the identification scores was evaluated. The results showed that N protein can be confidently identified using MALDI-TOF mass spectral profiles when the missed cleavage was larger than 0. Among the identification results, three allowed missed cleavage sites resulted in the best score and the score did not vary significantly when larger missed cleavage numbers were used. The results showed that the combination of rapid MALDI-TOF MS and peptide mass fingerprinting can be used for the detection of SARS-COV-2.

The example sulfate ester packed bed column and sample extraction, processing and analysis methods may be used to capture and identify other virus particles also. The peptides identified using MALDI-TOF MS and by Mascot database searching can also be used for distinguishing different strains of the SARS-COV-2 virus. For example, Eta/B.1.525. strain has a SD>Y transition at amino acid positions 2 and 3 from the natural variant from reference database UniProtKB-PODTC9 (NCAP_SARS2). Alpha/B.1.1.7. has a D>L transition at the amino acid position 3 from the natural variant. Eta/B.1.525. has a A>G transition at the amino acid position 12 from the nature variant. Omicron/B.1.1.529. has a P>L transition at the amino acid position 13 from the nature variant. The amino acid positions 31-33 are missing in Omicron/B.1.1.529. from the nature variant. Delta/B.1.617.2. has a D>G transition at the amino acid position 63 from the nature variant. Gamma/P.1. has a P>R transition at the amino acid position 80 from the nature variant. Delta/B.1.617.2 and Kappa/B.1.617.1 have a D>Y transition at the amino acid position 377 from the nature variant.

As used herein, a phrase referring to “at least one of” or “one or more of” a list of items refers to any combination of those items, including single members. For example, “at least one of: a, b, or c” is intended to cover the possibilities of: a only, b only, c only, a combination of a and b, a combination of a and c, a combination of b and c, and a combination of a and b and c. Unless otherwise specified in this disclosure, for construing the scope of the term “about” or “approximately,” the error bounds associated with the values (dimensions, operating conditions etc.) disclosed is ±10% of the values indicated in this disclosure. The error bounds associated with the values disclosed as percentages is ±1% of the percentages indicated. The word “substantially” used before a specific word includes the meanings “considerable in extent to that which is specified,” and “largely but not wholly that which is specified.”

Various modifications to the implementations described in this disclosure may be readily apparent to persons having ordinary skill in the art, and the generic principles defined herein may be applied to other implementations without departing from the spirit or scope of this disclosure. Thus, the claims are not intended to be limited to the implementations shown herein but are to be accorded the widest scope consistent with this disclosure, the principles and the novel features disclosed herein.

Additionally, various features that are described in this specification in the context of separate implementations also can be implemented in combination in a single implementation. Conversely, various features that are described in the context of a single implementation also can be implemented in multiple implementations separately or in any suitable subcombination. As such, although features may be described above in combination with one another, and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flowchart or flow diagram. However, other operations that are not depicted can be incorporated in the example processes that are schematically illustrated. For example, one or more additional operations can be performed before, after, simultaneously, or between any of the illustrated operations. In some circumstances, multitasking and parallel processing may be advantageous. Moreover, the separation of various system components in the implementations described above should not be understood as requiring such separation in all implementations, and it should be understood that the described program components and systems can generally be integrated together in a single product or packaged into multiple products.

### REFERENCES

Fennelly K. P., Martyny J. W., Fulton K. E., Orme I. M., Cave D. M., et al. (2004) Cough-generated aerosols of Mycobacterium tuberculosis: a new method to study infectiousness. Am J Respir Crit Care Med 169:604-609.
Hunt, J., “Exhaled breath condensate: An evolving tool for noninvasive evaluation of lung disease,” J. Allergy Clin. Immunol. 2002; 110:28-34.
Benjamin Patterson, Carl Morrow, Vinayak Singh, Atica Moosa, Melitta Gqada, Jeremy Woodward, Valerie Mizrahi, Wayne Bryden, Charles Call, Shwetak Patel, Digby Warner, Robin Wood, “Detection of Mycobacterium tuberculosis bacilli in bio-aerosols from untreated TB patients,” Gates Open Research 2018, 1:11.
Wood R., Morrow C., Barry C. E., III, Bryden W. A., Call C. J., Hickey A. J., et al.: Real-Time Investigation of Tuberculosis Transmission: Developing the Respiratory Aerosol Sampling Chamber (RASC). PLOS One. 2016; 11(1): e0146658.
Rachel C. Wood, Angelique K. Luabeya, Kris M. Weigel, Alicia K. Wilbur, Lisa Jones-Engel, Mark Hatherill, and Gerard A. Cangelosi, “Detection of Mycobacterium tuberculosis DNA on the oral mucosa of tuberculosis patients,” Sci. Rep. 5, 8668 (2015).

What is claimed is:

1. A passive exhaled breath aerosol particle capture system for capturing non-volatile organic particles in exhaled breath, the system including:
an exhaled breath capture module including:
an exhaled breath management chamber including:
a chamber inlet end configured to receive an exhaled breath tubing removably insertable into the chamber;
a chamber outlet end; and
a pressure relief port disposed near the chamber outlet end; and
an exhaled breath capture element including a packed bed column, the exhaled breath capture element removably connected to the chamber outlet end, wherein a gap of predetermined length is defined between an outlet end of the exhaled breath tubing when the exhaled air tubing is positioned in the exhaled breath management chamber and an inlet end of the exhaled breath capture element;
a passive CO₂ sensor disposed downstream of the exhaled breath capture element; and
one or more air filters removably connected to one or more of the pressure relief port or disposed downstream of the CO₂ sensor.

2. The passive exhaled breath aerosol particle capture system of claim 1, wherein the passive CO₂ sensor includes a colorimetric sensor.

3. The passive exhaled breath aerosol particle capture system of claim 1, wherein the packed bed column is kept in a wet state prior to capturing the non-volatile organic particles in exhaled breath.

4. The passive exhaled breath aerosol particle capture system of claim 1, wherein an exhaled breath aerosol particle capture efficiency associated with the passive exhaled breath aerosol particle capture system is greater than 99%.

5. The passive exhaled breath capture system of claim 1, wherein a pressure drop across the exhaled breath capture element is less than about 100 mbar.

6. The passive exhaled breath capture system of claim 1, wherein an outlet end of the exhaled breath capture element is disposed outside the exhaled breath management chamber.

7. The passive exhaled breath capture system of claim 1, wherein the chamber includes an adapter disposed at the inlet end of the chamber, the adapter defining an annular region between the adapter and the chamber, wherein the exhaled air tubing is removably inserted into the chamber through the chamber inlet end and through the adapter.

8. The passive exhaled breath capture system of claim 7, wherein the adapter includes a recess element configured to receive the outlet end of the exhaled air tubing, and wherein the recess element is configured to define the gap between the outlet end of the exhaled breath tubing and the inlet end of the exhaled breath capture element.

9. The passive exhaled breath capture system of claim 7, wherein the chamber inlet end includes a removable chamber cap, and wherein the adapter is removable from the chamber by opening the cap.

10. The passive exhaled breath capture system of claim 1, wherein the pressure relief port is disposed orthogonal to a longitudinal axis (A-A′) of the exhaled breath capture element when the capture element is removably connected to the chamber outlet end.

11. The passive exhaled breath capture system of claim 1, wherein a diameter of the inlet end of the exhaled breath capture element inlet is substantially equal to a diameter of the exhaled breath tubing.

12. The passive exhaled breath capture system of claim 1, wherein the packed bed column includes solid particles or beads including one or more of resins, cellulose, silica, agarose, or hydrated Fe₃O₄ particles.

13. The passive exhaled breath capture system of claim 1, wherein the packed bed column includes cellulose beads having sulfate ester functional groups on the surface.

14. The passive exhaled breath capture system of claim 1, wherein the packed bed column includes resin beads having C18 functional groups on the surface.

15. The passive exhaled breath capture system of claim 12, wherein the beads have an average diameter of between about 40 μm and about 120 μm.

16. The passive exhaled breath capture system of claim 12, wherein the beads are functionalized with one or more functional groups including immobilized on the surface of the particles, wherein the functional groups include at least one of C18 (octadecyl), octyl, ethyl, cyclohexyl, phenyl, cyanopropyl, aminopropyl, 2,3-dihydroxypropoxypropyl, trimethyl-aminopropyl, carboxypropyl, benzenesulfonic acid, propylsulfonic acid, an ion exchange phase, a polymer phase, antibodies, glycans, lipids, DNA, or RNA.

17. A system for analyzing non-volatile organic particles in exhaled breath, the system including:
the passive exhaled breath aerosol particle capture system of claim 1;
an extraction system configured to extract the captured non-volatile organic particles from the packed bed column in the exhaled breath capture element into one or more liquid samples; and
an analytical device configured to analyze the non-volatile organic particles in the one or more liquid samples.

18. The system of claim 17, wherein the extraction system includes means to flush the packed bed column with at least one solvent and produce the one or more liquid samples.

19. The system of claim 17, wherein the analytical device includes one or more of PCR, ELISA, rt-PCR, mass spectrometer (MS), MALDI-MS, ESI-MS, MALDI-TOFMS, or LC-MS/MS.

20. A method for analyzing non-volatile organic particles in exhaled breath, the method including:
capturing the non-volatile organic particles in exhaled breath using a passive exhaled breath aerosol particle capture system, the system including:
an exhaled breath capture module including:
an exhaled breath management chamber including:
a chamber inlet end configured to receive an exhaled breath tubing removably insertable into the chamber;
a chamber outlet end; and
a pressure relief port disposed near the chamber outlet end; and
an exhaled breath capture element including a packed bed column, the exhaled breath capture element removably connected to the chamber outlet end, wherein a gap of predetermined length is defined between an outlet end of the exhaled breath tubing when the exhaled air tubing is positioned in the exhaled breath management chamber and an inlet end of the exhaled breath capture element;
a passive CO₂ sensor disposed downstream of the exhaled breath capture element, wherein the CO₂ sensor includes a colorimetric sensor; and
one or more air filters removably connected to one or more of the pressure relief port or disposed downstream of the CO₂ sensor;
terminating the capturing operation when a color change is observed in the CO₂ sensor;
extracting the non-volatile organic particles from the packed bed column into one or more liquid samples by flushing the packed bed column with one or more solvents; and
analyzing the non-volatile organic particles in the one or more liquid samples.

21. The method of claim 20, further including maintaining the packed bed column in a wet state prior to capturing the non-volatile organic particles in the exhaled breath.

22. The method of claim 20, wherein the one or more solvents include one or more of acetonitrile (ACN), methanol, trifluoro acetic acid (TFA), or isopropanol (IPA), the remaining being water.

23. The method of claim 20, wherein analyzing includes analyzing the non-volatile organic particles in the one or more liquid samples using one or more of PCR, ELISA, rt-PCR, mass spectrometer (MS), MALDI-MS, ESI-MS, MALDI-TOFMS, or LC-MS/MS.
